(19) United States
(12) Patent Application Publication
(10) Pub. No.: US 2007/0259359 A1
(43) Pub. Date: Nov. 8, 2007

(54) Nanoelectronic Detection of Biomolecules Employing Analyte Amplification and Reporters

(76) Inventors: Mikhail Briman, Emeryville, CA (US); Eugene Tu, San Diego, CA (US); Christian Valcke, Orinda, CA (US)

Correspondence Address: O'MELVENY & MYERS LLP, 610 NEWPORT CENTER DRIVE, 17TH FLOOR, NEWPORT BEACH, CA, 92660, US

(21) Appl. No.: 11/695,401

(22) Filed: Apr. 2, 2007

Related U.S. Application Data
(63) Continuation-in-part of application No. 11/318,354, filed on Dec. 23, 2005.
(63) Continuation-in-part of application No. 11/212,026, filed on Aug. 24, 2005.
(63) Continuation-in-part of application No. 11/588,845, filed on Oct. 26, 2006.
(63) Continuation-in-part of application No. 11/488,456, filed on Jul. 18, 2006.
(60) Provisional application No. 60/901,538, filed on Feb. 14, 2007.
(60) Provisional application No. 60/850,217, filed on Oct. 6, 2006.
(60) Provisional application No. 60/789,022, filed on Apr. 4, 2006.
(60) Provisional application No. 60/748,834, filed on Dec. 9, 2005.
(60) Provisional application No. 60/738,694, filed on Nov. 21, 2005.
(60) Provisional application No. 60/730,905, filed on Oct. 27, 2005.
(60) Provisional application No. 60/668,879, filed on Apr. 5, 2005.
(60) Provisional application No. 60/657,275, filed on Feb. 28, 2005.
(60) Provisional application No. 60/639,954, filed on Dec. 28, 2004.
(60) Provisional application No. 60/629,604, filed on Nov. 19, 2004.
(60) Provisional application No. 60/604,293, filed on Aug. 24, 2004.
(60) Provisional application No. 60/850,217, filed on Oct. 6, 2006.
(60) Provisional application No. 60/773,138, filed on Feb. 13, 2006.
(60) Provisional application No. 60/748,834, filed on Dec. 9, 2005.
(60) Provisional application No. 60/730,905, filed on Oct. 27, 2005.
(60) Provisional application No. 60/700,953, filed on Jul. 19, 2005.

(51) Int. Cl. C12Q 1/68

(52) U.S. Cl. 435/6; 435/4

(57) ABSTRACT

Methods of detection of biomolecules are described, including methods of amplification of analyte target species and target reporters by analyte-triggered action of an enzyme such a nuclease, polymerase, and the like. Amplified target species (e.g., amplicons and reporters) are detectable by several embodiments of nanoelectronic sensors having aspects of the invention, and by alternative convention biomolecule detection methods.

### CROSS-REFERENCE TO RELATED APPLICATIONS

This application claims priority pursuant to 35 USC. §119(e) to the following U.S. Provisional Applications, each of which applications are incorporated by reference:
Ser. No. 60/901,538 filed Feb. 14, 2007 entitled “Electrochemical nanosensors for biomolecule detection”;
Ser. No. 60/850,217 filed Oct. 6, 2006 entitled “Electrochemical nanosensors for biomolecule detection”; and
Ser. No. 60/789,022 filed Apr. 4, 2006 entitled “Analyte amplification and reporters, and nanoelectronic detection of polynucleotides and other biomolecule”.

This application is a continuation-in-part of and claims priority to U.S. patent application Ser. No. 11/318,354 filed Dec. 23, 2005, entitled “Nanoelectronic sensor devices for DNA detection and recognition of polynucleotide sequences” (equivalent published as WO2006-071,895), which claims priority to (among other applications) U.S. Provisional Applications No. 60/748,834, filed Dec. 9, 2005; Ser. No. 60/738,694 filed Nov. 21, 2005; Ser. No. 60/730,905, filed Oct. 27, 2005; Ser. No. 60/668,879, filed Apr. 5, 2005; Ser. No. 60/657,275 filed Feb. 28, 2005; and Ser. No. 60/639954, filed Dec. 28, 2004.

This application is a continuation-in-part of and claims priority to U.S. patent application Ser. No. 11/212,026 filed Aug. 24, 2005, entitled “Nanotube sensor devices for DNA detection” (equivalent published as WO2006-024,023), which claims priority to (among other applications) U.S. Provisional Applications No. 60/629,604 filed Nov. 19, 2004; and Ser. No. 60/604,293 filed Aug. 24, 2004.

This application is a continuation-in-part of and claims priority to U.S. patent application Ser. No. 11/588,845 filed Oct. 26, 2006 entitled “Anesthesia Monitor, Capacitance Nanosensors and Dynamic Sensor Sampling Method”, which in turn claims priority to the follow U.S. provisional applications No. 60/850,217 filed Oct. 6, 2006; Ser. No. 60/773,138 filed Feb. 13, 2006; Ser. No. 60/748,834 filed Dec. 9, 2005 and Ser. No. 60/730,905 filed Oct. 27, 2005.

This application is a continuation-in-part of and claims priority to U.S. patent application Ser. No. 11/488,465 filed Jul. 18, 2006 entitled “Nanoelectronic Sensor With Integral Suspended Micro-Heater” (published as 2007-0045,756); which in turn claims priority to U.S. Provisional Application No. 60/700,953 filed Jul. 19, 2005.

This application is related in subject matter to the following U.S. patent applications, each of which applications are incorporated by reference:
Ser. No. 10/704,066 filed Nov. 7, 2003 entitled “Nanotube-Based Electronic Detection Of Biomolecules” (Publication 2004-0132,070);
Ser. No. 10/388,701 filed Mar. 14, 2003 “Modification Of Selectivity For Sensing For Nanostructure Device Arrays” (U.S. Pat. No. 6,905,655);
Ser. No. 10/345,783 filed Jan. 16, 2003, entitled “Electronic sensing of biological and chemical agents using functionalized nanostructures” (Publication 2003-0134,433); and
Ser. No. 10/280,265 filed Oct. 26, 2002 entitled “Sensitivity Control For Nanotube Sensors” (U.S. Pat. No. 6,894,359);
Ser. No. 10/177,929 filed Jun. 21, 2002 entitled “Dispersed Growth Of Nanotubes On A Substrate” (equivalent published as WO04-040,671).

### BACKGROUND OF THE INVENTION

1. Field of the Invention

The present invention relates to detector systems for biomolecules using nanoelectronic sensor devices.

2. Description of Related Art

Because base sequences in polynucleotides encode genetic information, the ability to read these sequences has contributed to many advances in biotechnology. This work has identified many important sequences that are linked to medical conditions. For example, the BRCA gene is usually present in women who suffer from breast cancer. To take advantages of these linkages in medical testing, various techniques have been developed to scan tissue samples for the occurrence of specific important sequences. These techniques have shortcomings that make them expensive, slow, and complex, so that they are unlikely to be useful for routine medical testing.

These techniques universally rely on the tendency of polynucleotides to hybridize. A strand of single-strand DNA (ssDNA) in solution readily combines with a complementary strand (cDNA) that contains an opposite base to pair with each base in the ssDNA. The result of this combination is double-stranded DNA (dsDNA), which can be processed and separated from ssDNA. Thus, to scan for a particular target sequence, an experimenter provides the appropriate cDNA as a probe sequence. If the target sequence is present in a sample, the target ssDNA will hybridize with the probe ssDNA to produce dsDNA, and this hybridization can be detected in some way.

Current methods have mainly focused on optical detection using fluorescence-labeled oligonucleotides with dyes, quantum dots or enhanced absorption of light by oligonucleotide-modified gold nanoparticles. For example, electrochemical detection of DNA hybridization may be performed using nanostructures as electrodes. However, electrochemical methods rely upon electrochemical behavior of the labels.

A first shortcoming arises because many methods of detecting this hybridization involve modification of the sample ssDNA before hybridization. Often, a fluorescent molecule is attached to the ssDNA. This molecule, known as a label, causes the ssDNA to be detected by optical instruments such as microscopes and spectrometers. Labeling is used to detect sample DNA after a hybridization step. If the target sequence is present in a labeled sample, the labeled ssDNA will be incorporated in labeled dsDNA, and the dsDNA will thus be detectable with optical instruments. Although the use of optical detection makes this approach convenient, the chemical reaction by which the DNA is labeled is expensive and time-consuming. A detection method which did not require labeling would significantly increase the usefulness of DNA scanning for routine medical tests.

A second problem results from the low sensitivity of traditional detection methods. Although some of these methods are sensitive to low concentrations of DNA, they require large absolute numbers of DNA molecules. In a medical application, only a few cells are usually available, and consequently only a few DNA molecules of the target sequence will be present in a sample. This problem has been ameliorated by the use of the polymerase chain reaction (PCR), which can amplify the quantity of target DNA a million-fold. Like labeling, PCR is a complex chemical reaction, which makes tests expensive and slow.

Certain work has utilized oligonucleotides in systems for detection of target analytes, generally using optical or fluorescent detection methods. See for example, US Published App. 2006-0040,286 entitled “Bio-Barcode Based Detection Of Target Analytes” and U.S. Pat. No. 6,750,016 entitled “Nanoparticles Having Oligonucleotides Attached Thereto And Uses Therefor”, each of which publication is incorporated by reference. However, these methods do not provide for an amplified detection response to a native target analyte (e.g., a non-PCR amplified analyte) and do not provide for electronic detection.

Certain work has explored amplified response to a native target analytes. See for example, H F Lee, Y Li, A W Wark and R M Corn, “Enzymatically Amplified SPR Imaging Detection of DNA by Exonuclease Ill Digestion of DNA Microarrays”, Analytical Chem., 77 5096-5100 (2005) and US Published App. 2005-0048,501 entitled “Method And Apparatus For Detection Or Identification Of DNA”, each of which publication is incorporated by reference. However, these methods do not provide a method of electronic detection.

Other biomolecules in addition to DNA are increasingly important in scientific understanding of biological processes and in practical applications such as medical diagnosis and treatment. Thus there is a need for detection of such biomolecules as proteins, polysaccharides, RNA and the like. Certain work has explored detection methods for such analytes as proteins. See for example, US Published App. 2006-0014,172 entitled “Aptamer-Nanoparticle Conjugates And Method Of Use For Target Analyte Detection”, which is incorporated by reference. However, these methods do not provide for an amplification of a target portion of a native target analyte and do not provide for electronic detection.

Thus, there is a clear need for a sensitive, fast and robust technique for detecting specific target biomolecules. Such a technique should operate without the use of PCR or other target analyte amplification methods and without the requirement for labeling of analyte species. Such a technique should provide of an amplified detection response, and should provide for electronic detection. In addition is desirable that such techniques provide for concentration and/or simplification or purification of an analyte sample, further increasing sensitivity and selectivity.

A salient objective of providing a method which avoids the necessity of labeling or amplification of analyte species and provides an electronic detection signal is to enable simplified, automated bio-detection capability conducive to fast and inexpensive point-of-care diagnostic applications, preferably that may be carried out non-laboratory clinical or home setting.

### SUMMARY OF THE INVENTION

Additional description and summary of devices and methods having aspects of the invention are described the following co-invented provisional and non provisional applications listed in the section above entitled “Cross-Reference To Related Applications”, each of which applications is incorporated by reference. The present application should be read together with the subject matter and descriptions of each of these applications. Among other things, these applications describe a number of alternative embodiments of nanoelectronic devices and methods for the label-free detection and identification of polynucleotides and other analyte species.

Nanoelectronic Detector Devices.

Nanostructures possess unique properties for sensor applications; in that they may be essentially one- dimensional so as to be extremely sensitive to electronic perturbations, are readily functionalized, and are compatible with many semiconducting manufacturing processes. Embodiments having aspects of the invention employ nanostructures which have properties heavily influence by the atoms are on the surface, thus providing a basis for sensitive electronic detection. Exemplary embodiments preferably include one or more carbon nanotubes, and more preferably one or more single-walled carbon nanotubes (SWNTs). Alternative device embodiments generally include an element including at least one nanostructure (“nanostructure element”) whose electronic properties are highly sensitive to interaction with a target analyte. One or more conducting elements may communicate with the nanostructure element to provide signal(s) for measurement of one or more device electronic properties which are influenced by the response of the nanostructure element to exposure to an analyte medium.

Generally the nanostructure element and conductors are disposed adjacent to a supporting substrate, which typically includes at least a dielectric surface (or surface coating) to provide electrical isolation of device elements. Substrates may be rigid or flexible, porous or non-porous, and may be generally planar or flat, or alternatively may have functional shapes, such as a tubular configuration, and may be of a number of alternative compositions, such as silicon oxide, silicon nitride, aluminum oxide, polyimide, and polycarbonate, and the like. In a number of examples described herein, the substrate includes one or more layers, films or coatings comprising such materials as silicon oxide, SIO2, Si3N4, and the like, upon the surface of a silicon wafer or chip.

Nanotube network devices. In embodiments of nanosensor devices, the nanostructured element may comprise a collective structure which includes a plurality of nanostructures, such as SWNTs or other nanotubes arranged to form a collective structure. In a number of preferred embodiments of nanosensor devices, the nanostructured element may advantageously comprise a random interconnected network of nanotubes (“nanotube network”) disposed on or adjacent a substrate, and communicating with at least one electrical lead. Nanotube networks may be made by such methods as chemical vapor deposition (CVD) with traditional lithography, by solvent suspension deposition, vacuum deposition, and the like. See for example, U.S. patent application Ser. No. 10/177,929 (corresponding to WO2004-040,671); U.S. patent application Ser. No. 10/280,265; U.S. patent application Ser. No. 10/846,072; and L. Hu et al., Percolation in Transparent and Conducting Carbon Nanotube Networks, Nano Letters (2004), 4, 12, 2513-17, each of which applications and publication is incorporated herein by reference.

Properties of the nanostructure elements (e.g., nanotube network) may by measured using one or more contacts. A contact includes a conducting element disposed such that the conducting element is in electrical communication with the nanostructure element, such as a nanotube network. For example, contacts may be disposed directly on a substrate surface, or alternatively may by disposed over a nanotube network. Electric current flowing in the nanotube network may be measured by employing at least two contacts that are placed within the defined area of the nanotube network, such that each contact is in electrical communication with the network.

Transistor embodiments. In some embodiments of the invention, an additional conducting element, referred to as a gate or counter electrode, is provided such that it is not in electrical communication with the nanostructured element (such as at least one nanotube), but such that there is an electrical capacitance between the gate electrode and the nanostructured element. Exemplary devices comprise field-effect transistors where the channel of the transistor comprises the nanotube(s), and the device may be referred to as a nanotube field effect transistors or NTFET. For example, the gate electrode is a conducting plane within the substrate beneath the silicon oxide. Alternatively, a gate or counter electrode may comprise a conductive layer disposed adjacent (e.g., under, above, beside), but electrically isolated from, the nanostructure element, such as a conductive polymeric material deposited on a flexible substrate. Resistance, impedance, transconductance or other properties of the nanotubes may be measured under the influence of a selected or variable gate voltage. Examples of such nanotube electronic devices are provided, among other places, in U.S. patent application Ser. No. 10/656,898, filed Sep. 5, 2003 (Publication 2005-0279987) and Ser. No. 10/704,066, filed Nov. 7, 2003 (Publication 2004-0132,070), both of which are incorporated herein, in their entirety, by reference.

A transistor device arrangement lends itself to measurement of the channel transconductance as a function of gate voltage (e.g., G/Vg signal). A transistor has a maximum conductance, which is the greatest conductance measured with the gate voltage in a range, and a minimum conductance, which is the least conductance measured with the gate voltage in a range. A transistor has an on-off ratio, which is the ratio between the maximum conductance and the minimum conductance. To make a sensitive chemical sensors, a nanotube transistor has an on-off ratio preferably greater than 1.2, more preferably greater than 2, and most preferably greater than 10.

Recognition of Analytes. Additional materials may be included in association with the nanostructure element (e.g., species or layers attached or absorbed upon one or more of the nanostructure element, the substrate, the conductor, and the like) to mediate the interaction of the device elements with the analyte medium, including target species, cross contaminants and the like. Such materials may include one or more of recognition layers or molecular transducers (such as the ssDNA oligomer probes in the following examples), catalyst materials, passivation materials, inhibition materials, protective materials, filters, analyte attractors, concentrators, binding species, and the like. Such materials and elements can function to improve selectivity, specificity and/or device service characteristics.

Polynucleotides Species. The invention provides an electronic sensor device with which to detect specific target sequences of polynucleotides. In certain embodiments, the sensor comprises nanostructured elements, (for example single and/or multiwalled carbon nanotubes and/or interconnecting networks comprising such nanotubes) which interact with polynucleotides so as to act as sensing elements. In the particular examples described in detail, the nanostructured elements comprise carbon nanotubes, and more particularly, randomly oriented networks of carbon nanotubes. In these examples, the nanotubes are modified before sensing by the adsorption of ssDNA probe sequences. No labeling of the DNA is required. Further, the invention provides a method for using the sensor device.

As used herein, “DNA” means polynucleotides. Examples of polynucleotides include, but are not limited to, deoxyribonucleic acid, ribonucleic acid, messenger ribonucleic acid, transfer ribonucleic acid, and peptide nucleic acid. The defining characteristics of polynucleotides are a chain of nucleic acids and a sequence of bases, each base chemically bonded to a nucleic acid and each base capable of pairing with an appropriate base on a matching sequence. Those skilled in the art will appreciate that other variations of polynucleotides may be produced which share these defining characteristics. Accordingly, a “single-strand DNA”, referred to hereafter as “ssDNA”, may be a single strand of deoxyribonucleic acid, ribonucleic acid, or any other polynucleotide as described above. A “double-strand DNA”, referred to hereafter as “dsDNA” or duplex polynucleotide, may be a double strand of any polynucleotide described above. “Complimentary DNA”, referred to hereafter as “cDNA”, may be any strand of a polynucleotide described above which is a single-strand sequence complimentary to an already referenced single-strand sequence.

The ssDNA in a particular sensor device may be selected to be cDNA for a particular target sequence. The target sequence is the sequence of bases that the sensor device is intended to detect. The cDNA for the target sequence is known as the probe sequence. Once a target sequence is specified, a quantity of DNA with the probe sequence must be obtained. A variety of techniques are known for synthesizing DNA with specified sequences and for synthesizing DNA complementary to a given sequence. Those skilled in the art will have knowledge of these techniques. Further, appropriate cDNA or other polynucleotide to make a probe specific to a desired target sequence can generally be obtained from known commercial suppliers serving the biotechnology industry.

A sensor device may be used by exposing the nanotube network to a solution containing sample ssDNA. The network should be exposed to the solution for a period of time long enough for hybridization to occur. This period of time depends on the concentration of the sample DNA, the quantity of the solution, the temperature of the room, the pH of the solution, and other variables. Those skilled in the art are familiar with the effect of these variables on DNA hybridization and are capable of choosing an appropriate period of time, solution composition, temperature and other conditions of hybridization without undue experimentation.

It should be noted that, with respect to all the described sensor embodiments, that the occurrence, speed and specificity of polynucleotide hybridization depends on various conditions. In each of these hybridization schemes, the binding energy of the dsDNA can be challenged through stringency techniques. This can be done through temperature increases or buffer changes, for example sodium hydroxide.

Additional stringency controls may include various ionic constituents of the hybridization medium, such as sodium or magnesium ions. Alternatively or additionally, a voltage may be applied to elements of the sensor (e.g., a nanotube network) before, during and/or after hybridization to influence polynucleotide behavior. For example, a polynucleotide such as cDNA has a phosphate-based backbone which typically is ionized in the hybridization medium so as to carry a localized negative charge. Selectively charged sensor elements may be used to provide an attractive or repulsive stringency factor, for example, to destabilize a SNP-mismatched probe hybrid relative to a corresponding fully-matched probe hybrid (e.g., during incubation or during a rinse process).

Through variations in stringency, it is possible to differentiate binding of strands with complete or incomplete complementary base pairs. Changes in electrical properties of the nanotubes in response to the stringency process allow discrimination of single base mismatches (SNP), among other things. One of ordinary skill in the art will be able to vary the hybridization conditions so as to tune the operation of certain embodiments of the sensors of the invention to obtain a selected degree of sensitivity to complete and less-than-complete hybridization of the target sequence.

For example, in an assay to discriminate between a DNA sample which is homozygous for a particular allele, on the one hand, and an otherwise comparable sample which is heterozygous for this allele, the stringency of the hybridization conditions may be adjusted (e.g. by variation in temperature) so as to produce a distinctly different device measurement response between the homozygous and heterozygous samples.

In the case of each of the sensor embodiments having aspects of the invention, these sensors may be constructed in arrays, e.g., arrays of transistor sensors functionalized for a plurality of different target DNA fragments. See U.S. application Ser. No. 10/388,701 entitled “Modification Of Selectivity For Sensing For Nanostructure Device Arrays” (publication 2003-0175,161), incorporated by reference herein.

Electrochemical detector embodiments. In some embodiments of the invention, nanostructured elements, such as an electrode including a carbon nanotube network, may be employed to detect electrochemical interactions of analyte target species and/or reporters described herein, so as to permit measurement of presence or concentration of one or more analytes in a sample. See, for example, the detection methods and devices as described in co-invented U.S. patent application Ser. No. 60/901,538 filed Feb. 14, 2007 and Ser. No. 60/850,217 filed Oct. 6, 2006, each entitled “Electrochemical nanosensors for biomolecule detection”. Each of these applications is incorporated herein by reference.

Capacitance detector embodiments. In some embodiments of the invention, detectors including nanostructured capacitive elements may be employed to detect electrochemical interactions of analyte target species and/or reporters described herein, so as to permit measurement of presence or concentration of one or more analytes in a sample. See, for example, the detection methods and devices as described in co-invented U.S. patent application Ser. No. 11/588,845 filed Oct. 26, 2006 entitled “Anesthesia Monitor, Capacitance Nanosensors and Dynamic Sensor Sampling Method”, which claims priority to Ser. No. 60/850,217 filed Oct. 6, 2006; Ser. No. 60/773,138 filed Feb. 13, 2006; Ser. No. 60/748,834 filed Dec. 9, 2005 and Ser. No. 60/730,905 filed Oct. 27, 2005. Each of these applications is incorporated herein by reference.

Alternative detection methods, labels, markers and devices. Although the exemplary embodiments described in detail focus on reporters advantageously detectable by electronic signals independent of chemically or optically active labels or markers, alternative methodology is possible without departing from the spirit of the invention.

For example, reporter fragments, species or amplicons derived by any of the amplification methods described herein may contain particular markers, labels or detection enhancers configured to effect any one of a number of different reporter or analyte detection methods known in the art, such as fluorescent markers, quenching groups, mass alteration, optical detection, spectroscopy, electrophoretic mobility alteration, receptor affinity alteration and the like. Likewise, reporter fragments, species or amplicons derived by any of the amplification methods of the invention may be configured to participate in secondary biomolecular reactions so as to have a detectable effect, e.g., by catalytic effects, or by triggering the vulnerability of a fluorescent probe to independent enzymatic attack or reconfiguration, or the like.

Non-PCR Reporter Amplification.

One aspect of the invention provides methods of electronically detecting a quantity of a reporter molecule, such as a specific DNA oligomer, following the amplified release of the reporter molecule in response to a much smaller quantity of target analyte. In certain embodiments having aspects of the invention, enzymes having specific activity on polynucleotides may be employed to achieve a non-PCR amplification of a reporter moiety which is indicative of the presence of a selected analyte and which is electronically detectable by use of nanosensor having aspects of the invention. Such embodiments may be described as having non-cyclical amplification in that the amplification process does not require a elaborate system of control for cyclically varying reaction conditions, as is the case in PCR for example.

In a first embodiment having aspects of the invention, a probe assembly comprises a substrate, such as a magnetic bead, a titration well, or the like, to which is bound one or more probe oligonucleotides. For example, a bead substrate may support a plurality of distinct and separately-functional probe oligonucleotides on the bead surface, so as to form a multi-probe assembly.

Each such probe oligonucleotide includes a nanocode sequence portion and a capture sequence portion. The nanocode sequence portion is selected to be complementary to a corresponding sequence on a reporter oligonucleotide. In preparation of the probe assembly, the reporter oligonucleotide is hybridized, under suitable conditions, to the nanocode sequence. The capture sequence portion is selected and configured to be complementary to a corresponding sequence on a target analyte polynucleotide (target sequence). Probe oligonucleotide with capture sequence and nanocode may be manufactured to specification by known methods, and attached to substrates such as magnetic beads or other immobilization surface by known methods.

In operation, each target analyte polynucleotide is bound by a probe to form an analyte/probe complex (there may be an excess of probes,. e.g. many probes per magnetic bead). In certain embodiments (e.g. an magnetic bead or other substrate) immobilization of the substrate with bound analyte/probe complex permits purification or rinsing of the complex, thus simplifying the sample or lysis mixture. In a preferred embodiment, exonuclease (added after rinsing) has specific activity so as to degrade only capture sequence and nanocode of probe assembly portion of an analyte/probe complex, but not target analyte polynucleotide or reporter oligonucleotide (or pristine probes). Exonuclease activity releases both target analyte polynucleotide and reporter oligonucleotide, so that: (a) analyte polynucleotide is free to react with additional pristine probes; and (b) reporter oligonucleotides accumulate in reaction buffer to a high concentration. Accumulated (amplified) reporter oligonucleotides in simplified media may be electronically detected without significant cross reactivity by proprietary nanoelectronic detectors, as described herein.

In a first embodiment of analyte-triggered release of reporter described below, the example employs an exonuclease having a specific activity for degradation (e.g. by hydrolysis of phosphate backbone bonds) of double stranded polynucleotides having a non-protruding 3′ terminal end (e.g. blunt or recessed 3′). It should be understood that alternative apparatus and methods may be configured to employ other exonuclease activity, such as a “mirror image” example, wherein the 3′ and 5′ configurations of polynucleotides and oligonucleotides are reversed, and the exonuclease has 5′ end specificity. Still other alternatives are possible without departing from the spirit of the invention.

Alternative embodiments can employ 5′ reactive exonucleases, sticky-end exonulceases, etc; and can use other immobilization surfaces (polymer or Si surfaces, other bead types, etc.), cleavable linker between probe, media volume reduction, bead filtration, and the like.

In additional alternative embodiments other enzymes may be employed under selected conditions to achieve selective exonuclease activity. For example, various types of DNA polymerase may be employed in a media deprived of nucleotides tri-phosphates, so as to favor “proof-reading” activity at the expense of polymerase activity, thus functioning as a nuclease.

In additional alternative embodiments, the constituent elements of the probes may include polynucleotides with synthetic base analogs, such as locked nucleic acids (e.g., LNA Oligos or nucleic acids including a 2′-O, 4′-C methylene bridge, such as are available from Sigma-Aldrich Corp.). LNA oligomers may be used to block or limit exonuclease activity in selected portions of the probe molecules, and to control stability of hybridized duplexes.

In additional alternative embodiments, antibody composite probes and/or aptamer-based probes for protein, polysaccharide or other biomolecule target analytes may be employed to permit detection of non-polynucleotide analytes.

Preferably, the exemplary detection methods and devices having aspects of the invention include concentration and simplification steps to increase sensor response, sensitivity and/or selectivity. Probes may be bound or immobilized for separation or rinsing steps by attachment to any solid support or substrate suitable for binding the oligomers. Examples of suitable substrate materials include, but are not limited to, glass, plastics, polyethylene, cellulose, polymethacrylate, latex, rubber, fluorocarbon resins , metals, and the like. The substrate material may be configured as a slide, well or other enclosure, or alternatively as a particle, such as a microsphere or microbead. Paramagnetic coatings can render bead magnetically responsive. Conjugating or complexing substances may be employed to bind oligonucleotides or other probe species to substrates, e.g. avidin or an avidin derivatives, suitable for binding biotin or biotin derivatives.

Magnetic bead probe conjugation and separation/immobilization techniques are well known and the constituents and accessories are commercially available. See for example, Invitrogen Corporation, http://www.invitrogen.com/ (formerly Dynal Biotech) of Carlsbad, Calif. See also the separation methods and devices described in US Published Application 2005-0147,822 entitled “Process”; U.S. Pat. No. 5,512,439 entitled “Oligonucleotide-linked Magnetic Particles And Uses Thereof”; U.S. Pat. No. 6,994,971 entitled “Particle Analysis Assay For Biomolecular Quantification”; and U.S. Pat. No. 5,851,770 entitled “Detection Of Mismatches By Resolvase Cleavage Using A Magnetic Bead Support”; each of which publications are incorporated by reference. Magnetic beads of sizes on the order of 1 micron or less are available, optionally pre-treated with binding constituents such as covalently bound streptavidin (e.g., for binding to avidin-treated oligonucleotides), bacterial wall proteins (e.g., for binding to antibodies), and the like.

Alternative separation/immobilization modalities for particulate probe substrates may be used, such as electrophoresis separation. Beads or particles having positive charged groups (e.g., amino) or negative charged groups (e.g., carboxylic acid) may be separated or immobilized by electric field forces. In other alternatives, particulate or bead substrates may be separated by filtration from a reaction medium.

In one embodiment of a method of detecting an analyte polynucleotide in a sample, the method comprises:
(a) providing at least a first probe assembly, the first probe assembly comprising: (i) a substrate; (ii) at least one probe polynucleotide including a proximal 5′-terminal nucleotide and a distal 3′-terminal nucleotide, the probe polynucleotide bound adjacent the proximal 5′-terminal nucleotide to the substrate, the probe polynucleotide comprising at least one nanocode nucleotide sequence portion, and a capture nucleotide sequence portion having a nucleotide sequence complementary to a corresponding target nucleotide sequence of the analyte polynucleotide; (iii) at least a first reporter polynucleotide having a nucleotide sequence complementary to the nanocode sequence of the probe polynucleotide, the first reporter polynucleotide hybridized to the nanocode sequence to form a duplex portion with the probe polynucleotide; and(iv) the probe polynucleotide so configured that the capture nucleotide sequence portion and 3′-terminal nucleotide extends distally from the first reporter polynucleotide duplex portion;
(b) contacting the first probe assembly with a sample which putatively contains the analyte polynucleotide, under conditions effective to allow for binding of the target nucleotide sequence of the analyte polynucleotide to the capture nucleotide sequence portion of the probe polynucleotide to form a duplex first probe/analyte complex, the first probe/analyte complex including: (i) the first probe assembly; (ii) at least one analyte polynucleotide; and (iii) the analyte polynucleotide extending distally to form either of (1) a blunt distal 5′-terminal end or (2) a protruding distal 5′-terminal end; (c) contacting the first probe/analyte complex with an exonuclease, the exonuclease having 3′-to-5′ exo-deoxyribonuclease activity including specific binding to double-stranded DNA followed by selective hydrolysis of the 3′ terminated strand of the DNA duplex;
(d) maintaining conditions effective to allow binding of the exonuclease to the first probe/analyte complex and effective to allow hydrolysis of the distally 3′ terminated strand of the first probe/analyte complex by the exonuclease to an extent that the analyte polynucleotide and at least the first reporter polynucleotide are released from the first duplex probe/analyte complex; and
(e) determining the presence of the analyte polynucleotide in the sample by detecting the presence of at least the first reporter polynucleotide released by the exonuclease hydrolysis.

An alternative embodiment of a method of detecting an analyte polynucleotide in a sample comprises the mirror-image method relative to that of the paragraph above, in which 3′ and 5′ terminal ends are reversed throughout the definition of the method, so that:
(a) the substrate is conjugated adjacent a proximal 3′ end of the probe polynucleotide;
(b) the distal end of the first probe/analyte complex includes the analyte polynucleotide extending distally to form either of (1) a blunt distal 3′-terminal end or (2) a protruding distal 3′-terminal end;
(c) the exonuclease has 5′-to-3′ exo-deoxyribonuclease activity including specific binding to double-stranded DNA followed by selective hydrolysis of the 5′ terminated strand of the DNA duplex;
(d) the method includes maintaining conditions effective to allow hydrolysis of the distally 5′ terminated strand of the probe/analyte complex to release the analyte polynucleotide and at least the first reporter polynucleotide; and
(e) the method includes determining the presence of the analyte polynucleotide in the sample by detecting the presence of the reporter polynucleotide released by the exonuclease 5′-to-3′ hydrolysis.
   Exponential Non-PCR Target Amplification.

One aspect of the invention provides methods of amplifying target analyte polynucleotide related species, so as to provide enhanced detection scope for rarified samples or minute quantities of sample analyte. Alternative amplification methods and apparatus may include:
(1) Homologous reporters. Amplifying detectable species representative of target analyte polynucleotide presence in a sample by analyte-triggered release of corresponding synthetic target oligonucletides from amplifier reagent duplex species via exonuclease-mediated reactions, where the released synthetic target oligonucletides comprise detectable copies of portions of the target analyte polynucleotide sequence (homologous or quasi-homologous analog oligonucleotides); and/or
(2) Non-homologous reporters. Amplifying detectable species representative of target analyte polynucleotide presence in a sample by analyte-triggered release of corresponding synthetic target oligonucletides from amplifier reagent duplex species via exonuclease-mediated reactions, where the released synthetic target oligonucletides comprise sequences not closely resembling the target analyte polynucleotide sequence (non-homologous analog oligonucleotides).

In a first embodiment of amplifier method and apparatus described below, the example employs an exonuclease having a specific activity for degradation (e.g. by hydrolysis of phosphate backbone bonds) of double stranded polynucleotides having a non-protruding 3′ terminal end (e.g. blunt or recessed 3′). It should be understood that alternative apparatus and methods may be configured to employ other exonuclease activity, such as a “mirror image” example, wherein the 3′ and 5′ configurations of polynucleotides and oligonucleotides are reversed, and the exonuclease has 5′ end specificity. Still other alternatives are possible without departing from the spirit of the invention.

Dual target amplifiers. In an embodiment having aspects of the invention, the sequences of two distinct portions of a single-stranded analyte polynucleotide are selected, conveniently designated Target A and Target B. An amplifier reagent may be compounded so as to include at least two species of amplifier:
(1) Amplifier “A” including an oligonucleotide which has a first capture sequence complementary to Target B (Capture B′) and a second capture sequence complementary to Target A (Capture A′).
(2) Amplifier “B” including an oligonucleotide which has a first capture sequence complementary to Target A (Capture A′) and a second capture sequence complementary to Target B (Capture B′).

Duplex amplifier with companion. Each of the pristine Amplifier A and Amplifier B in the reagent comprises a duplex or double stranded form, in which the amplifier oligonucleotide is hybridized with a companion oligonucleotide configured to produce a duplex which lacks any non-protruding 3′ end, so as to be protected from exonuclease activity in its pristine form. The companion oligonucleotide includes a sequence (synthetic target) which mimics a corresponding target sequence of the analyte molecule, as follows:
(1) the companion oligonucleotide of Amplifier “A” includes a synthetic target sequence the same or similar to Target A of the analyte (Synthetic Target A”), the Synthetic Target A” being hybridized in the pristine reagent to the second capture sequence Capture A′; and
(2) the companion oligonucleotide of Amplifier “B” includes a synthetic target sequence the same or similar to Target B of the analyte (Synthetic Target B″), the Synthetic Target B″ being hybridized in the pristine reagent to the second capture sequence Capture B′.

Amplifier hybridizing with target. Each of Amplifier A and Amplifier B is configured (in the pristine duplex form) to expose a constituent capture sequence so that the capture sequence (under conditions effective to promote polynucleotide hybridization) may hybridize with the corresponding target sequence of the analyte polynucleotide, so as to form a corresponding Amplifier/Analyte Complex. Thus, for example:
(1) the first capture sequence Capture B′ of Amplifier A binds to Target sequence B of the analyte, and
(2) the first capture sequence Capture A′ of Amplifier B binds to Target sequence A of the analyte.

Note that each Amplifier/Analyte Complex may include hybridization of either one or both of Amplifier A and Amplifier B with each analyte polynucleotide. The process need not proceed in synchronized form as the amplifiers types are configured to react in the describe process independently of each other.

Amplifier/Analyte Complex enzymatic degradation. In any one of these cases, the selection of target sequence locations and/or configuration of the amplifier provides that the Amplifier/Analyte Complex (either having Amplifier A, an Amplifier B or both) includes an exposed non-protruding 3′ end of each amplifier oligonucleotide, so that exonuclease present in the reagent (or added separately) may initiate degradation of the amplifier oligonucleotide. As degradation of the amplifier oligonucleotide continues, both capture sequences are removed, so as to release the target analyte from the amplifier oligonucleotide, and to release from the amplifier oligonucleotide the corresponding companion oligonucleotide having a synthetic target sequence, as follows:
(1) the amplifier oligonucleotide of Amplifier A (in an Amplifier/Analyte Complex) is degraded to release the Target sequence A of the analyte and to release the Synthetic Target A″ of the companion oligonucleotide; and
(2) the amplifier oligonucleotide of Amplifier B (in an Amplifier/Analyte Complex) is degraded to release the Target sequence B of the analyte and to release the Synthetic Target B″ of the companion oligonucleotide.

Thus, for each original analyte molecule, the above describe amplification process releases both the original un-degraded analyte polynucleotide molecule (with original Target A and Target B), two single-stranded synthetic targets derived from amplifier reagent (Synthetic Target A and Synthetic Target B), and any exonuclease enzyme which mediated the reaction.

Subsequent amplification of released targets. In the presence of additional pristine Amplifier A and Amplifier B and in the presence of exonuclease, each of these target sequences (synthetic or native analyte) may participate in further processes of amplification as described above, because each amplifier is configured to hybridize (under conditions effective to promote polynucleotide hybridization) with a synthetic target with the same effect as with the native target:
(1) the first capture sequence Capture B′ of a pristine Amplifier A binds to Synthetic Target B″ of the released companion oligonucleotide of a previously-degraded Amplifier B to form an all-synthetic Amplifier/Target Complex, which in turn is degraded by exonuclease activity to release both the original Synthetic Target B″ and an additional companion oligonucleotide including a Synthetic Target A″, and
(2) the first capture sequence Capture A′ of a pristine Amplifier B binds to Synthetic Target A″ of the released companion oligonucleotide of a previously-degraded Amplifier A to form an all-synthetic Amplifier/Target Complex, which in turn is degraded by exonuclease activity which release both the original Synthetic Target A″ and an additional companion oligonucleotide including a Synthetic Target B″.

Each “cycle” of the amplification produces (releases) both an original target and a corresponding new target, each of which can trigger additional amplification events. As a result, it may be seen that during the continued amplification process, and while the medium includes sufficient amplifier reagent and exonuclease, that the population of original analyte polynucleotide tends remains constant while the population of Synthetic Target A″ and Synthetic Target B″ tends to grow exponentially. Note the use of the term “cycle” in this context does not imply the particular cyclic variation of environmental conditions such as temperature that is typically employed in PCR, and the conditions of the medium may be selected and controlled so that both polynucleotide hybridization and enzymatic degradation activity are promoted simultaneously and continuously.

Multistage amplifiers. An embodiment having aspects of the invention includes a method of introducing a reporter species into a medium where the medium includes a biomolecular template species, the method comprising a non-PCR, template-triggered, enzyme-activated release of the reporter species from a probe assembly having a binding affinity for the template species. The method including in any operative order the steps of:
(a) providing at least a first probe assembly, the first probe assembly including: (i) at least a first probe strand having a capture nucleotide sequence which provides a selective binding affinity for a target portion of the template species; (ii) at least one first reporter species including a binding portion having a polynucleotide sequence configured to hybridize with a corresponding binding portion of the probe strand; (iii) wherein the first probe assembly includes at least one first probe strand and at least one first reporter species hybridized to comprise a polynucleotide duplex probe assembly; and (iv) wherein the duplex probe assembly is configured so as to have at least one enzyme-initiation site suited to promote the action of a selected enzyme having nuclease activity sufficient to degrade all or a portion of the probe strand so as to release the first reporter species from the probe assembly, the enzyme-initiation site being formed in the event that the capture nucleotide sequence binds with all or part of the selected target portion of the template species so as to form a template-probe complex;
(b) contacting the medium with at least one first probe assembly under conditions effective to promote binding of the selected target portion of the template species to the capture nucleotide sequence portion of the probe polynucleotide to form a first template-probe complex having an enzyme-initiation site;
(c) contacting the medium with at least the selected enzyme under conditions effective to promote nuclease activity of the enzyme at an enzyme-initiation site, so that in the event that a first template-probe complex has been formed in step (b), the first probe strand is degraded so as to release the first reporter species from the probe assembly.

The amplification of this embodiment may be carried out so as to have multiple stages by additional steps whereby the first the first reporter species includes a template portion which is configured to act as a target template for at least one second non-PCR, template-triggered, enzyme-activated release of a second reporter species from a second probe assembly having a binding affinity for the template portion of the first reporter species. This may be extended to have a third or more stage in further reporter species are released.

In a biomolecule detection method, the multistage amplification method may be used to detect analytes such as polynucleotides, proteins, polysaccharides, and other biomolecular species. In embodiments, the first probe assembly includes capture nucleotide sequence complementary to a target sequence of the template (e.g., template includes a DNA strand). In alternative embodiments, the first probe assembly includes an aptamer as a capture nucleotide sequence, the aptamer having an affinity for a template target portion (e.g., template includes a polypeptide or polysaccharide target portion).

In an embodiment, the biomolecular template species includes an analyte species in a sample, the first reporter species is configured to be directly or indirectly detectable when released from the probe assembly, the method further including the steps of:
(d) directly or indirectly detecting the released first reporter species;
(e) determining at least a presence or concentration of the analyte species in the sample based on the direct or indirect detection of the first reporter species.

For example, the first reporter species is configured to be directly detectable when released from the first probe assembly, such as by including a detection portion having a detectable polynucleotide sequence or a detectable label group (single stage detection). Likewise, the first reporter species may indirectly detectable when released from the first probe assembly, via additional amplification stages in which further reporter species are detectable.

Hairpin probe assembly. Further exemplary embodiments having aspects of the invention may eliminate separate capture and reporter portions of the probe assembly. In an embodiment, the first probe strand and the first reporter species comprise a co-linear polynucleotide strand, wherein portions of the co-linear polynucleotide strand are configured to self-hybridize when not in association with the target portion of the template species so as to be protected from degradation by the selected enzyme.

A more complete understanding of the devices and methods having aspects of the invention will be afforded to those skilled in the art, as well as a realization of additional advantages and objects thereof, by a consideration of the following detailed description of the preferred embodiment.

### BRIEF DESCRIPTION OF THE DRAWINGS

The following is a list which summarizes the drawings and figures herein:

FIGS. 1A through 1H illustrate an exemplary embodiment of methods and apparatus having aspects of the invention of electronically detecting a quantity of a reporter molecule, such as a specific DNA oligomer, following the amplified release of the reporter molecule in response to a much smaller quantity of target analyte.

FIGS. 2A through 2H illustrate an exemplary embodiment of methods and apparatus having aspects of the invention for electronic detection which provide a non-linear or exponential analyte-responsive amplification.

FIGS. 3A and 3B summarize two examples of the process of amplification, both as shown in FIGS. 2A-H, one example employing two analyte targets (dual target) and an alternative example employing a single analyte target (single target).

FIG. 3C is a plot showing the data of Tables 1 and 2, depicting the increase or amplification of synthetic targets by the single target scheme and dual target scheme.

FIGS. 4A through 4B illustrate alternative apparatus and methods for reporter amplification and purification.

FIGS. 5A through 5E illustrate an alternative exemplary method and apparatus embodiment having aspects of the invention which provide exponential analyte-responsive amplification including a pre-amplification purification process.

FIGS. 6A through 6C illustrate one exemplary method and apparatus embodiment having aspects of the invention which provide exponential analyte-responsive amplification followed by removal of exonuclease from the media under analysis.

FIGS. 7A-F shows an example of a reporter probes having aspects of the invention and including with internal blocking groups which stop the processing of exonuclease at the sequence location of the blocking group.

FIGS. 8A and 8B illustrate one exemplary method and apparatus embodiment having aspects of the invention including a multi-analyte assay with multiple reporter types and a matrix detector cell.

FIGS. 8A and 8B illustrate one exemplary method and apparatus embodiment having aspects of the invention including a multi-analyte assay with multiple reporter types and a matrix detector cell.

FIGS. 9A to 9C illustrate one exemplary method having aspects of the invention employing a probe assembly including an aptamer portions for detection on biomolecules.

FIG. 10 comprises views 10a-10i which depict an exemplary embodiment of a two-stage method of target-initiated amplification so as to produce amplicons and/or reporter species.

FIG. 11 is a plot showing exemplary data corresponding to the method of FIG. 10

FIGS. 12A-12C depict an exemplary embodiment of a three-stage method of target-initiated amplification so as to produce amplicons and/or reporter species. FIG. 13 is a plot showing exemplary data corresponding to the method of FIGS. 12A-C.

FIGS. 14A-14C illustrate an example of single-target amplification method having aspects of the invention (See, e.g., FIG. 3A) utilizing fluorescent detection, wherein:

FIG. 14A is a diagram of the amplifier-analyte complex;

FIG. 14B is a photograph of a electrophoretic gel showing results and amplification product of the method, and

FIG. 14B is a negative version of the photograph of FIG. 14B, which provides a more distinct indication of the data in a photo-reproducible image suitable for patent illustration.

FIGS. 15A-15C illustrate an example of a “hairpin” type probe assembly.

### DETAILED DESCRIPTION OF THE PREFERRED EMBODIMENT

The present invention provides a nanotube sensor device that detects a target DNA sequence. The device requires no labeling of the target DNA and responds electronically to the presence of the target DNA. Exemplary embodiments are described below.

Analyte-Triggered Reporter Amplification.

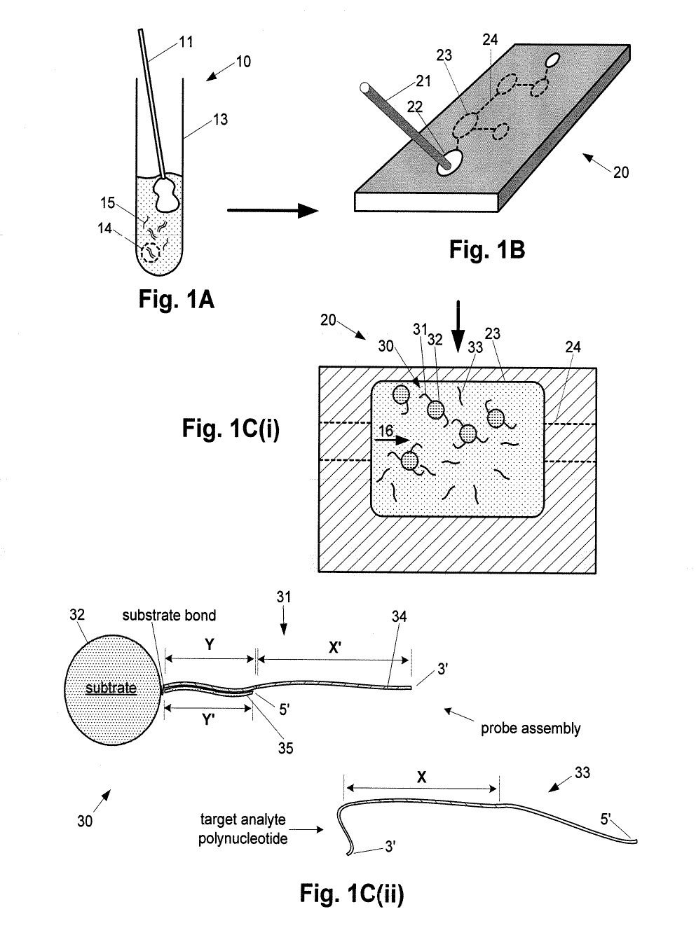

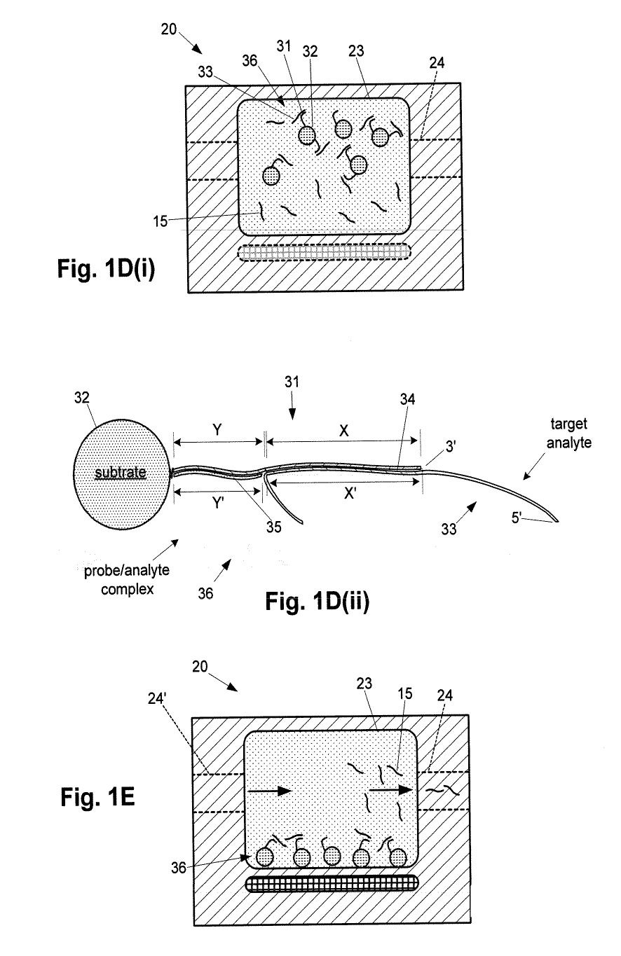

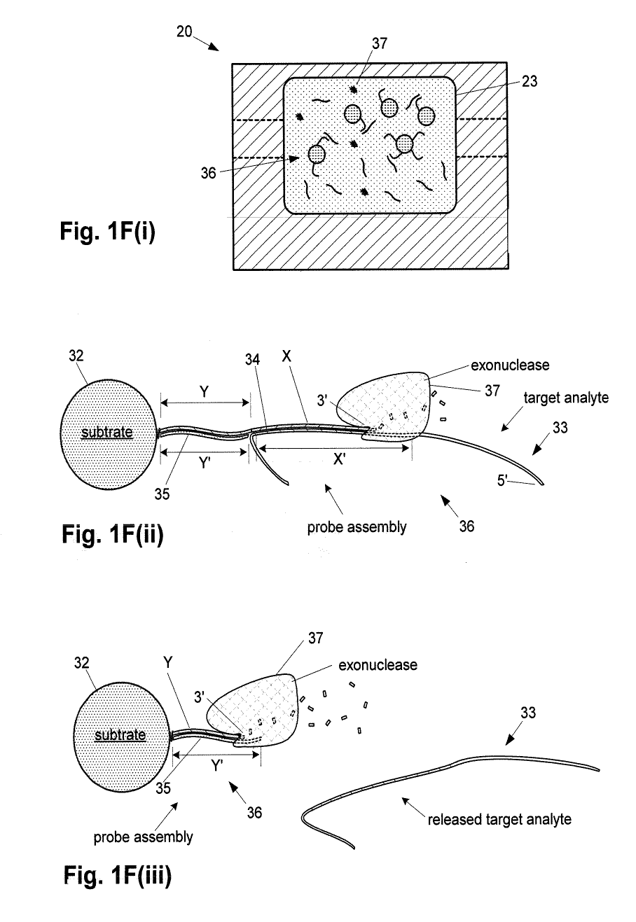

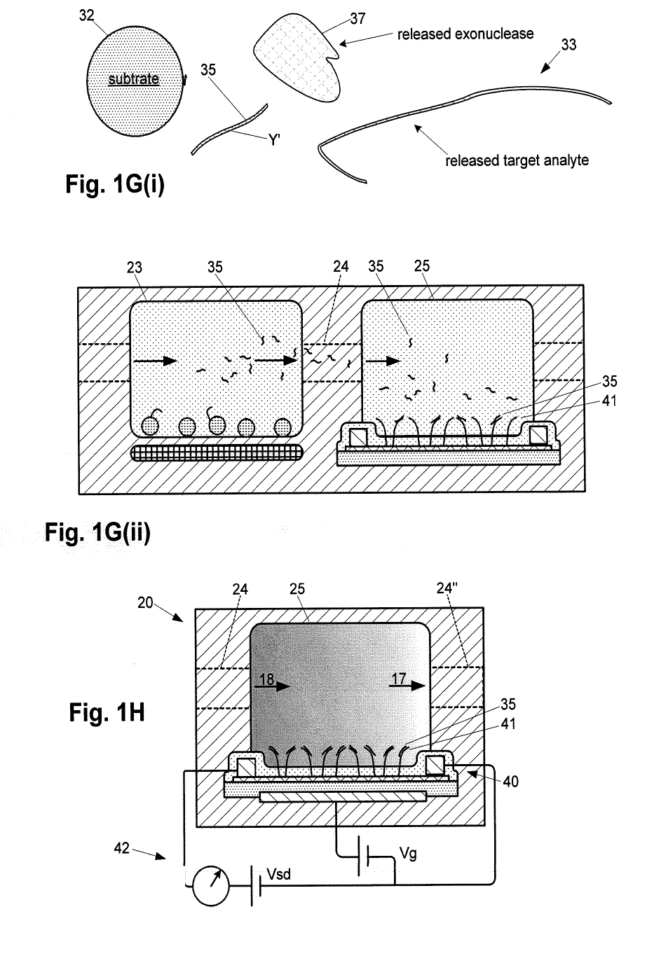

FIGS. 1A through 1H illustrate an exemplary embodiment of methods and apparatus having aspects of the invention of electronically detecting a quantity of a reporter molecule, such as a specific DNA oligomer, following the amplified release of the reporter molecule in response to a much smaller quantity of target analyte. In certain embodiments having aspects of the invention, enzymes having specific activity on polynucleotides may be employed to achieve a non-PCR amplification of a reporter moiety which is indicative of the presence of a selected analyte and which is electronically detectable by use of nanosensor having aspects of the invention. Such embodiments may be described as having non-cyclical amplification in that the amplification process does not require a elaborate system of control for cyclically varying reaction conditions, as is the case in PCR for example.

FIG. 1A shows a figurative example of sample collection and preparation step 10, depicting a sample applicator 11 (e.g., a throat swab or the like) inoculating a lysis buffer or media 12 in a container 13 to release cellular or viral material 14, which is in turn lysed to release sample polynucleotide material 15.

FIG. 1B shows a figurative example of sample processing in a fluidic system 20 comprising an applicator 21 (e.g., a pipette) applying lysed sample media to an inlet port 22, which communicates with a pattern of conduits 24 (e.g., of a microfluidic cartridge) connecting enclosure cells or vessels 23.

FIGS. 1C(i) and 1C(ii) show sample media 16 comprising polynucleotide material 15 as contained in vessel 23 containing one or more probe assemblies 30, each probe assembly comprising a substrate 32 attached to one or more capture probes 31.

Note in this example the substrate 32 comprises a magnetic bead, but other particulate and non-particulate capture substrates (e.g., non-magnetic particles, fixed plates, enclosure walls, titration well, or the like) may be employed. For example, a bead substrate may support a plurality of distinct and separately-functional probes 31 on the bead surface, so as to form a bead-centered multi-probe assembly 30.

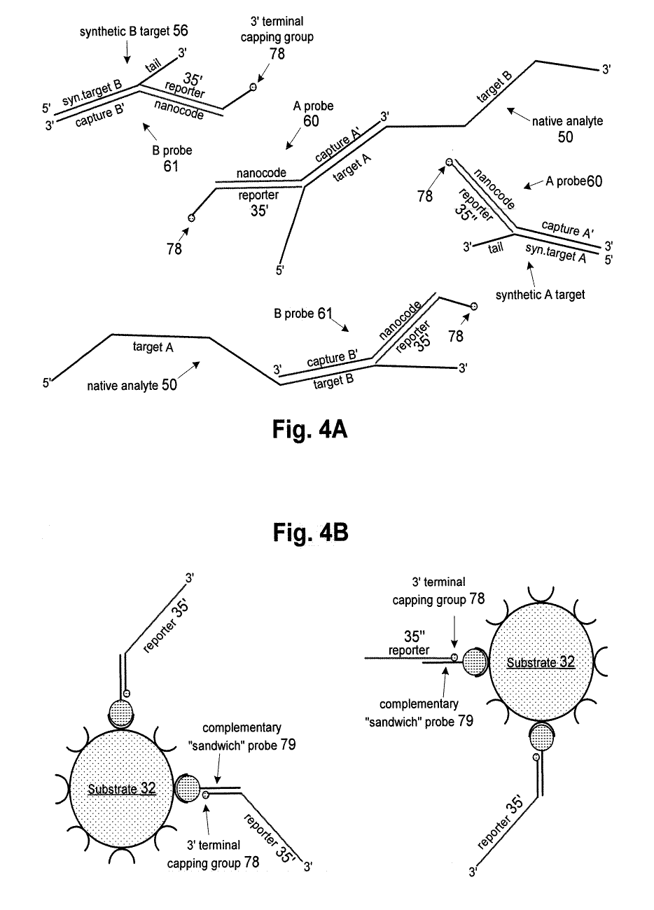

FIG. 1C(ii) shows detail of a probe assembly 30 and target analyte 33. The target analyte is a single strand polynucleotide having a target nucleotide sequence X between 3′ and 5′ terminal strand ends. Probe assembly 30 comprises a capture probe polynucleotide 34 bonded to substrate 32, for example by a biotin-streptavidin bond. Alternatively, a probe such as probe 30 may be bound to a substrate by a complementary “sandwich” type oligonucleotide structure, for example the structure of complementary “sandwich” probe 79 described below with respect to FIG. 4B.

In this example, the probe polynucleotide 34 is bound to substrate 32 adjacent a proximal 5′ terminal nucleotide, and comprises a nanocode sequence Y and a capture sequence X′, which is complementary to target sequence X on analyte polynucleotide 33. The probe 31 also includes one or more reporter oligonucleotides 35 having a complementary sequence Y′ which in the assembled probe is hybridized to the nanocode sequence Y of the probe polynucleotide 34. Note that although FIG. 1C(ii) depicts the nanocode sequence Y as being proximal (relative to substrate 32) with respect to the capture sequence X′, this need not be so.

Probe oligonucleotide 34 with selected capture and nanocode sequences as well as reporter oligonucleotide 35 may be manufactured to specification by known methods, such as by commercially available synthetic services. Likewise, biotinilated oligonucleotides are commercially available having designated base sequences, and substrates may be treated with streptavidin by know methods. Other suitable protocols for attaching polynucleotides to substrates are known, such as by use of cell surface ligands and the like. Reporters 35 may be hybridized to polynucleotide 34 under suitable conditions to complete probe assembly 30.

It should be understood that, unless defined more distinctly for a specific purpose, the terms polynucleotide and oligonucleotide are generally used herein interchangeably. While the selected lengths of particular sequences is relevant to the hybridization properties of the molecules, the principals of the invention apply to a wide range of sequence lengths and molecular weights without departing from the spirit of the invention. Where the context or usage makes it clear that a polynucleotide or oligonucleotide is being referenced, such species may also be referred to as a “molecule” or “species”, or by molecule function, such as “analyte” or “reporter”, without loss of clarity.

In this example, each probe oligonucleotide includes a nanocode sequence portion and a capture sequence portion. The nanocode sequence portion is selected to be complementary to a corresponding sequence on a reporter oligonucleotide. In preparation of the probe assembly, the reporter oligonucleotide is hybridized, under suitable conditions, to the nanocode sequence. The capture sequence portion is selected and configured to be complementary to a corresponding sequence on a target analyte polynucleotide (target sequence). Probe oligonucleotide with capture sequence and nanocode may be manufactured to specification by known methods, and attached to substrates such as magnetic beads or other immobilization surface by known methods.

FIGS. 1 D(i) and 1 D(ii) shows the vessel or cell 23 of fluidic system 20 in which target analyte polynucleotides 33 have hybridized with the probe nucleotides 34 of probes 31 to form one or more probe/analyte complexes 36, the probe/analyte complex 36 comprising the probe assembly 30 bound to the analyte polynucleotide 33 by hybridization of the target sequence X of the analyte 33 with the capture sequence X′ of at least one probe polynucleotide 34. FIG. 1 D(ii) shows detail of a probe/analyte complex 36, depicting the target analyte 33 having a distal 5′ end extending beyond and overlapping the distal 3′ end of the probe polynucleotide 34.

FIG. 1E shows the one or more substrates 32 immobilized (or alternatively fixedly mounted) to permit one or more optional rinsing or washing steps in which sample medium 16 with un-reacted species 15 is removed and replaced by a reaction medium 17, leaving the one or more probe/analyte complexes 36 (and any unreacted probes or probe assemblies) in the vessel 23. In this example one or more substrates are magnetic beads immobilized by controllable magnet 18.

Where probe assembly 30 comprises a plurality of probes 31, a plurality of analytes 33 may bind to respective probes 31 of each such probe assembly 30. In a detection system 20, there may advantageously be provided probe assembly or assemblies 30 having in total an excess of probes 31 such that analyte 33 presented in a sample is efficiently bound by available capture probes 31 in the step of FIGS. 1D. Rinsing/washing of the step of FIG. 1E may then advantageously remove non-target polynucleotides and other contaminants present in the initial sample or lysis mixture, so as to minimize the likelihood or extent of non-specific binding and cross reactivity during detection.

FIGS. 1F(i), 1F(ii), 1F(iii) and 1G(i) show the treatment of the probe/analyte complexes 36 with a exonuclease 37, in this example the exonuclease 37 having a specific activity to degrade (e.g., hydrolysis of phosphate bonds) duplex hybridized polynucleotides strands having a blunt or recessed (non-protruding) 3′ end exposed. In this example, the non-protruding 3′ distal end of probe polynucleotide 34 is attacked and degraded progressively. In a preferred embodiment, specific activity of the exonuclease (added after optional rinsing) acts to degrade only capture sequence and nanocode of probe assembly portion of an analyte/probe complex, but not target analyte polynucleotide 33 or reporter oligonucleotide 35 (or any pristine or unreacted probes 31). It should be understood that nucleases, as with other enzymes, have variable processivity depending on type and environment, and what for convenience is illustrated as a continuous process of degradation may be discontinuous or episodic as nuclease diffuses on or off the substrate oligonucleotide. Likewise, nucleases may have both primary and secondary activity (such as endonuclease activity), and one of ordinary skill in the art will be able to adjust concentrations and conditions to favor desired exonuclease activity.

As noted above, alternative embodiments may have probes configured to reverse the order of proximal and distal terminal strand ends (3′ for 5′) and employ exonucleases with a corresponding reversed activity (specific to non-protruding 5′ duplex ends). Likewise, in alternative embodiments, enzymes systems other than specific endonucleases and having multiple activity modes can be employed where non-specific activity is controlled or non-interfering. For example, various types of DNA polymerase may be employed in a media deprived of nucleotide tri-phosphates, so as to favor “proof-reading” activity at the expense of polymerase activity, thus functioning as a nuclease.

FIG. 1F(iii) shows the degradation of probe molecule 34 proceeding so as to release the analyte molecule 33 by removal of capture sequence X′.

FIG. 1G(i) shows the completed degradation of probe molecule 34 so as to release the reporter molecule 35 by removal of nanocode sequence Y.

Note that the analyte molecule 33 released in the step of FIG. 1 F(iii) is free to diffuse within medium 17 and hybridize with any remaining unreacted (“pristine”) probes 31 to form additional probe/analyte complexes 36. Such subsequently formed complexes 36 are in turn likewise vulnerable to exonuclease degradation, so as to release additional reporters 35. The provision of an excess of probes 31 permits an advantageous amplification process whereby each analyte molecule 33 is permitted to generate the sequential release of a plurality of reporter molecules from the analyte-stimulated degradation of a corresponding plurality of probes 31.

Thus the accumulation of a comparatively high concentration of reporter oligonucleotides in the reaction buffer, which has optionally been previously purified or simplified by rinsing, permits a very low initial concentration of analyte in the sample to be electronically detected without significant cross reactivity by nanoelectronic detectors, as described herein.

FIG. 1G(ii) shows the flow of accumulated reporters 35 to an adjacent detector cell 25 having one or more nanoelectronic sensors 40 as described herein (see incorporated patent applications and further sensor embodiments described below). Sensor 40 has a sensitivity for reporter probe 35, for example by having one or more detector probe 41 comprising a oligonucleotide complementary to at least a portion of the sequence of reporter 35. Alternatively, the detector 40 may be included in the reaction cell 23.

As shown FIG. 1H, the hybridization of reporter 35 with detector probe 41 results in a specific detectable change in the electronic properties detector 40 so as to permit the determination of the presences of reporter 35 by operation of measurement circuitry 42:
a. reporters hybridize with detector probes.
b. optional step: reaction buffer may be replaced by measurement buffer (e.g., to inhibit nuclease activity at detector).
c. signal acquired (e.g., transistor or capacitive properties, etc.)
d. reporters detected.

Optionally, the reaction medium 17 may be removed (e.g., replaced with a measurement buffer 18) following detector probe hybridization, prior to signal acquisition by circuitry 42.

Exponential Non-PCR Amplification.

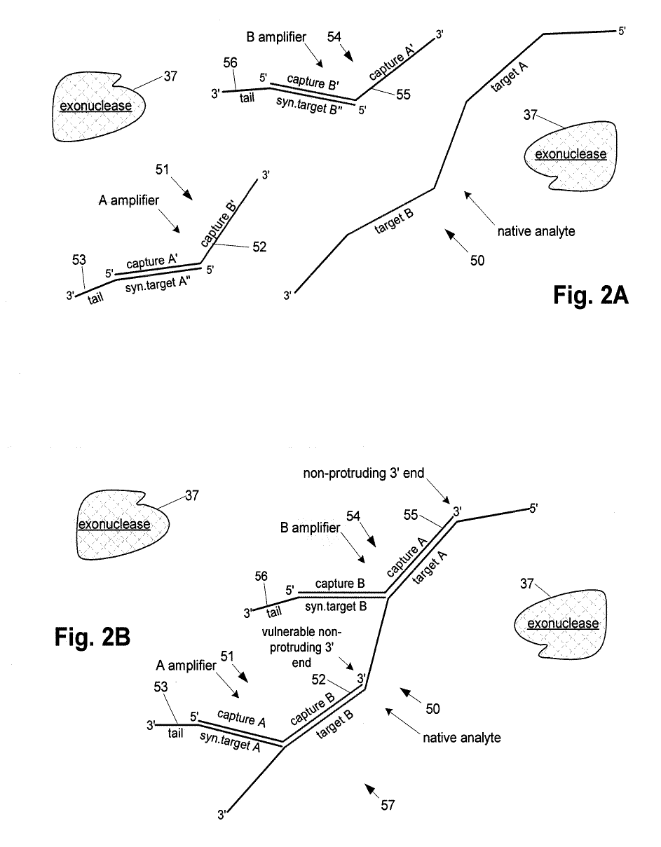

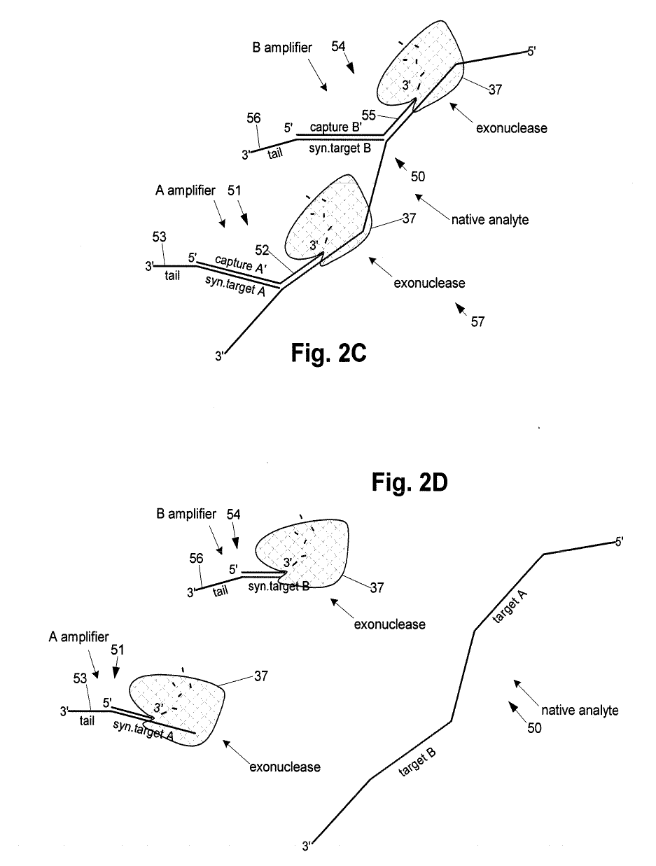

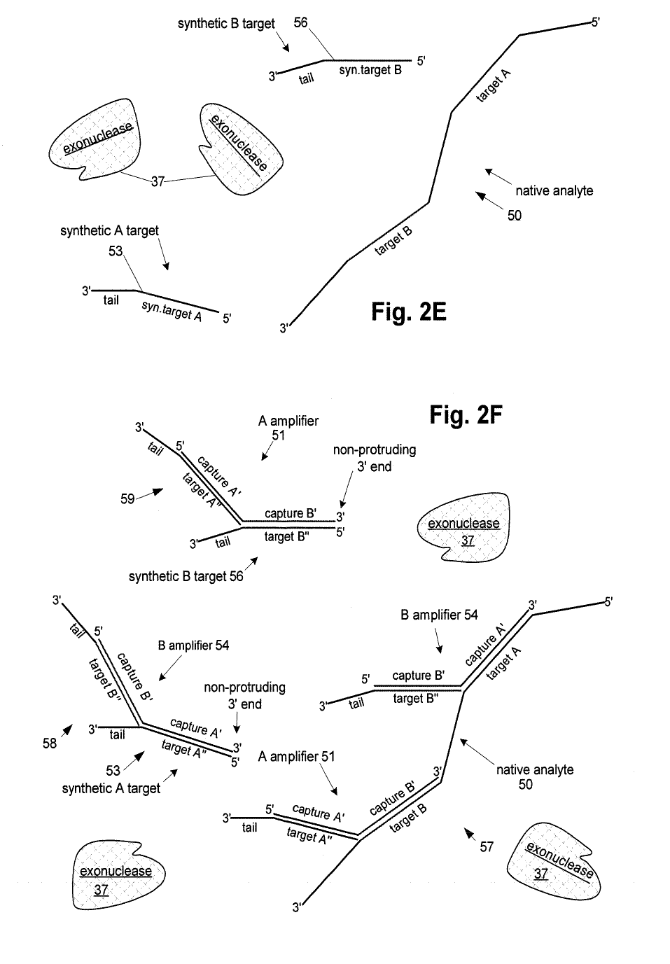

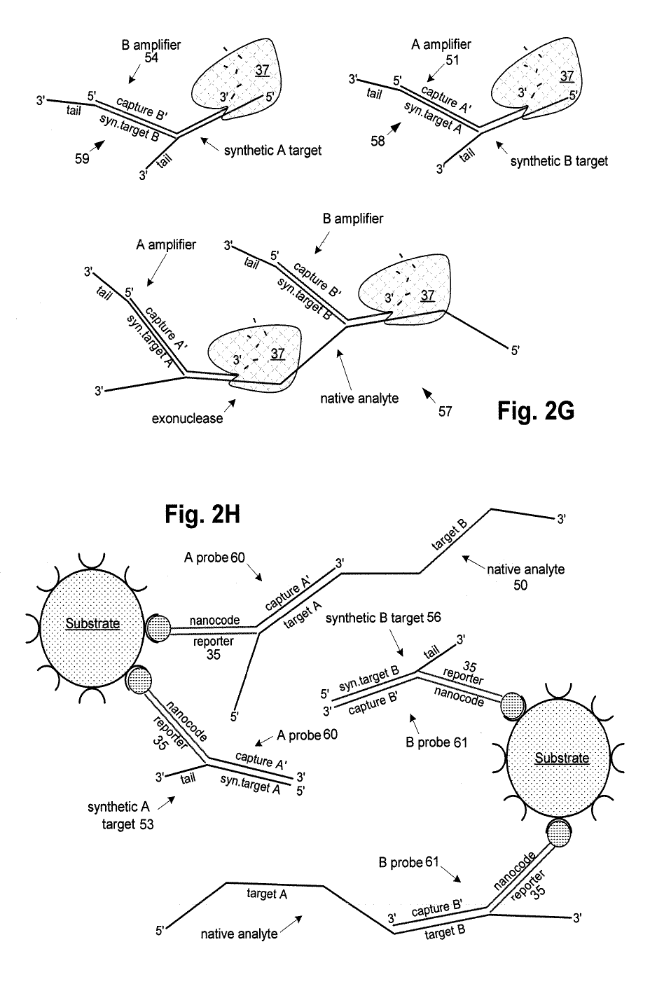

FIGS. 2A through 2H illustrate an exemplary embodiment of methods and apparatus having aspects of the invention (among a number of alternative methods having aspects in common) for electronic detection which provide a non-linear or exponential analyte-responsive amplification (additional non-sample targets are made available or “revealed” in response to analyte presence in sample, e.g., so as to participate in stimulating reporter release). A method such as in FIGS. 2A-H may be employed independently or in combination with methods such as exemplified by FIGS. 1A-H, which may be characterized as providing linear analyte-responsive amplification (e.g., only target analyte present in initial sample participates in stimulating reporter release).

In certain embodiments an initial analyte-responsive enzyme-mediated reaction releases “synthetic” (i.e., pre-prepared natural or synthesized oligonucleotide not present in sample) oligonucleotide targets which participate in additional enzyme-mediated reactions which in turn result in the release of further synthetic targets. The synthetic oligonucleotide targets are provided in a duplex form configured to be protected from exonuclease activity in the absence of analyte polynucleotide in the sample.

In some embodiments, the “synthetic” targets may mimic natural sequences of the target analyte, and in alternative embodiments, the synthetic targets may be entirely non-natural, or combinations of natural and non-natural sequences. The non-linear or exponential proliferation of such targets in the reaction medium may be employed to stimulate the release of reporter probes, such as by the methods shown in FIGS. 1A-H.

Note that the term “amplification” is used herein in a somewhat different sense that the usage of the term in PCR. In an example of a PCR assay method, new copies of sequences may be created via polymerase activity, triggered by binding of primers to analyte in a sample. In the examples of methods and apparatus having aspects of the invention described herein, copies of selected target sequences are pre-synthesized, and pre-compounded as amplifier groups, for example, as a reagent material The release of pre-synthetic targets is specifically analyte-triggered upon use of the reagent material in an assay, so that the synthetic targets are representative of the presence of analyte species in the sample. The proliferation of released representative molecules in a reaction medium constitutes, in effect, an amplified signal representative of analyte presence.

FIG. 2A shows the sample medium including a target analyte polynucleotide 50 comprising a target sequence A (target A), and also a target sequence B (target B). The sample is shown treated with a mixture including exonuclease 37, “A” amplifier 51 and “B” amplifier 54. Note that the sample medium may be subjected to optional purification steps prior to amplifier treatment, as described herein. In this example, the exonuclease 37 may be the same or similar enzyme as in the example of FIG. 1A-H, with specific activity for non-protruding 3′ duplex strand ends (note that 3′/5′ reverse-direction method alternatives similar to those described above are likewise possible). The analyte 53 includes two selected non-overlapping sequences designated target A and target B respectively.

“A” amplifier 51 comprises a synthetic oligonucleotide 52 having a sequence B′ (capture B′) which is complementary to the natural target sequence B (target B), and also having a sequence A′ (capture A′) which is complementary to the target sequence A (target A).

“A” amplifier 51 further comprises a second oligonucleotide 53 having a sequence A″ (synthetic target A″) which is complementary to the natural target sequence A (target A). The second oligonucleotide 53 has a “tail” sequence at its 3′ terminal end so as to configure oligonucleotide 53 so that it has a protruding 3′ “sticky end” (i.e., does not have a blunt or recessed 3′ end) so that it does not form a point of attack for the 3′ specific exonuclease 37. Alternatively, capping groups may be substituted (by know methods) for the “tail” sequence for this purpose.

The oligonucleotide 53 is bound to oligonucleotide 52 by hybridization of synthetic target A″ to capture A′. Note that “A” amplifier 51 is so designated because it can “reveal” a sequence (synthetic target A″) that is the same as or similar to the natural target sequence A of analyte 50.

Conversely, “B″ amplifier 54 comprises a synthetic oligonucleotide 55 having a sequence A′ (capture A′) which is complementary to the natural target sequence A (target A), and also having a sequence B′ (capture B′) which is complementary to the target sequence B (target B). “B” amplifier 54 further comprises a second oligonucleotide 56 having a sequence B″ (synthetic target B″) which is complementary to the natural target sequence B (target B). The second oligonucleotide 56 has a “tail” sequence (or other capping group) at its 3′ terminal end. The oligonucleotide 56 is bound to oligonucleotide 55 by hybridization of synthetic target B″ to capture B′. Note that “B” amplifier 54 is so designated because it can “reveal” a sequence (synthetic target B″) that is the same as or similar to the natural target sequence B of analyte 50.

Note the terms “natural” and “synthetic” are used in this context only to distinguish between the target analyte or “natural” polynucleotide putatively present in a sample, and the “synthetic” oligonucleotide provided in an amplifier reagent for purposes of carrying out an exemplary assay method. The target analyte or “native” polynucleotide of the sample often is, but need not be, from a natural source. The reagent amplifier oligonucleotides conveniently may be, but do not need to be, synthetically made.

FIG. 2B shows the elements of FIG. 2A, in which the “A” amplifier 51 and “B” amplifier 54 have reacted so as to hybridize with the target B and target A sequences respectively of analyte polynucleotide 50, so as to form a analyte/amplifier complex 57. Note that although both the “A” and “B” amplifiers 51, 54 are illustrated as having reacted simultaneously with a single analyte molecule 50, this need not be the case, as the amplification process described herein related to each amplifier may proceed independent of the other amplifier. For example, each amplifier may bind to a separate analyte molecule.

FIG. 2C shows analyte/amplifier complex 57 of FIG. 2B, in which the non-protruding 3′ terminal ends of the oligonucleotide 52 of “A” amplifier 51 and the comparable non-protruding 3′ terminal ends of the oligonucleotide 55 of “B” amplifier 54 have begun to be degraded by action of a pair of exonuclease 37 (not that the action of the enzyme at both sites need not be simultaneous).

FIG. 2D shows analyte/amplifier complex 57 of FIG. 2C, in which the activity of the exonuclease 37 has proceeded so as to remove the capture B′ sequence of oligonucleotide 52 and the capture A′ sequence of oligonucleotide 55, so as to release the native analyte polynucleotide 50.

FIG. 2E shows analyte/amplifier complex 57 of FIG. 2C, in which the activity of the exonuclease 37 has proceeded so as to remove the capture A′ sequence of oligonucleotide 52 and the capture B′ sequence of oligonucleotide 55, so as to release the corresponding oligonucleotides 53 and 56 thus exposing in a single stranded form (“revealing”) the corresponding synthetic targets A″ and B″.

FIG. 2F shows the elements of FIG. 2E, in which both the released native analyte polynucleotide 50 and the released synthetic targets A and B (oligonucleotides 53 and 56 with their corresponding targets A″ and B″ respectively) have reacted with additional “A” amplifier 51 and “B” amplifier 54, so as to form additional hybridized duplex forms: and additional amplifier/analyte complex 57, a synthetic A target/amplifier complex 58, and a synthetic B target/amplifier complex 59.

FIG. 2G shows the elements of FIG. 2F, in which further activity of exonuclease 37 has begun degrading the exposed non-protruding 3′ ends of complexes 57, 58 and 59 in the manner shown in FIGS. 2D-2E above, so as to release additional synthetic targets A and B, as well as the native analyte 50 and the previously released synthetic targets A and B.

FIG. 2H shows the elements of FIG. 2G (original accumulated native analyte 50 and accumulated synthetic targets A and B being treated with capture/reporter probe assemblies generally similar to those described with respect to the methods exemplified by FIGS. 1A-H. In this particular example, two distinct probe assemblies are employed, a first having probe 60 specific to the target sequence A (in either the native analyte 50 or the synthetic target A (53); and a second having probe 61 specific to the target sequence B (in either the native analyte 50 or the synthetic target B (56). The method and apparatus for release of reporter molecules 35.

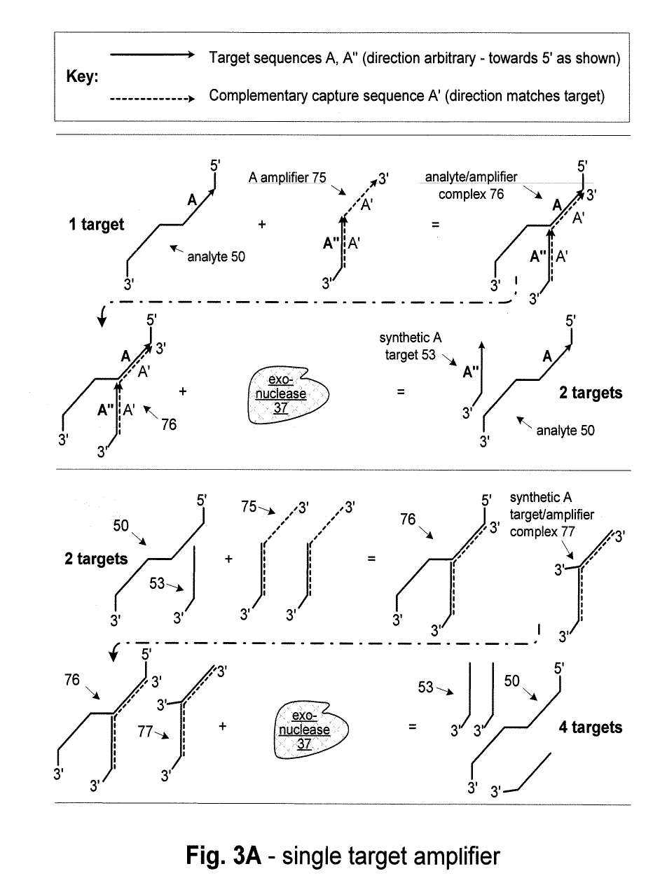

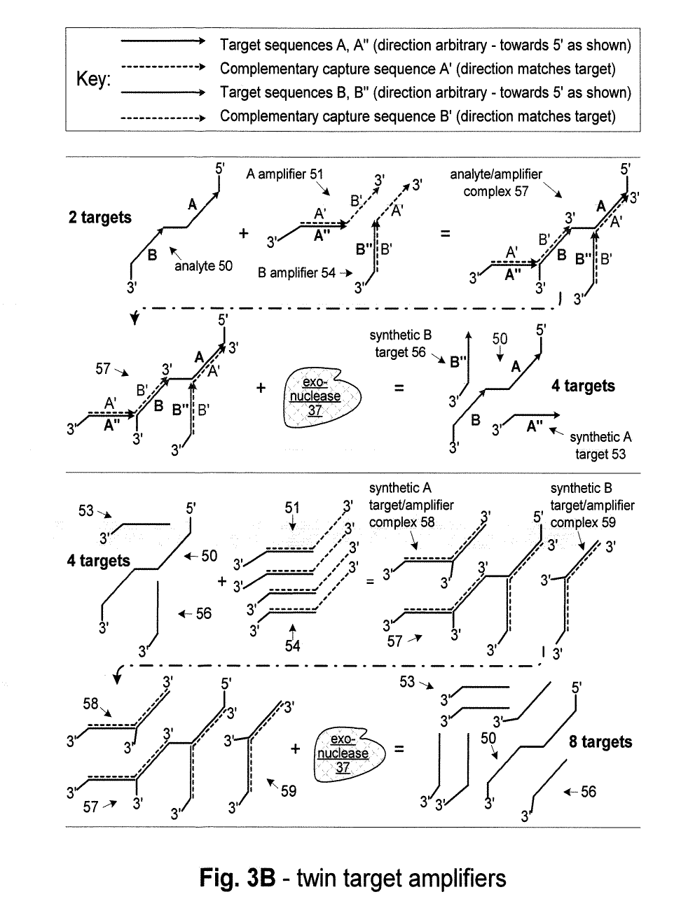

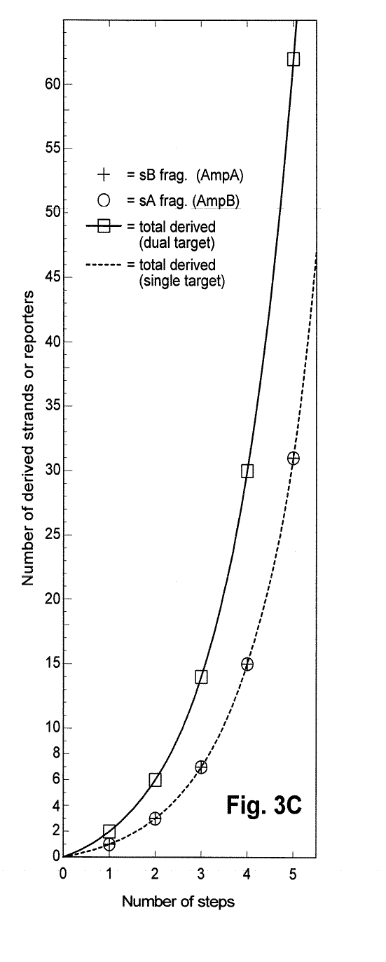

FIGS. 3A and 3B summarize two examples of the process of amplification, both as shown in FIGS. 2A-H (employing two analyte targets) and an alternative example employing a single analyte target.

Single target. FIG. 3A schematically illustrates the outcome of an amplification method using a single analyte capture sequence to trigger the release of synthetic targets. Analyte 50 has a single selected target sequence portion A (target A). Amplifier 75 is similar to amplifier 51 shown in FIG. 2A, except that both capture sequences are complementary to target A.

In a first phase, a single analyte 50 reacts with a single amplifier 75 to produce an analyte/amplifier complex 76. Exonuclease 37 degrades complex 76 to release analyte 50 and synthetic A target 53. Thus 2 targets are released.

In a second phase, both analyte 50 and synthetic A target 53 react with two additional amplifiers 75 to produce an analyte/amplifier complex 76 as well as a synthetic A target/amplifier complex 77. Exonuclease 37 degrades both complex 76 and complex 77 to release analyte 50 and three synthetic A targets 53. Thus 4 targets are released.

It may be seen that, since each target sequence that hybridizes with an additional amplifier group at each phase or step, the result is an exponential increase in released target presence. Table 1 shows the results of five phases or steps of this method.

TABLE 1
Single target

| | | derived |
|---|---|---|
| | original | fragments |
| Step (s) | targets | sA |
| initial | 1 | 0 |
| 1 | 1 | 1 |
| 2 | 1 | 3 |
| 3 | 1 | 7 |
| 4 | 1 | 15 |
| 5 | 1 | 31 |

Dual target. In contrast to FIG. 3A, FIG. 3B schematically illustrates the outcome of an amplification method using a more than one analyte capture sequence to trigger the release of synthetic targets. In this example the method is the same as illustrated in FIGS. 2A-H, but other alternatives are possible. For example Target A and Target B need not be selected to be in the sample analyte polynucleotide, but may be selected to be portions of co-analytes, for example, where the amplified target response is triggered by the simultaneous presence of both co-analytes in a sample. This is particularly useful, for example, in a selective assay in which a positive result is defined as the presence of more than one marker (e.g., derived from more than one fragment or molecule). As described also in FIGS. 2A-H, analyte 50 includes two selected target sequence portions that can react with two distinct amplifiers 51, 54 to produce complex 57. Exonuclease reaction then degrades the amplifiers to release both analyte 50 (two targets) and two distinct synthetic targets 53, 56. In the subsequent phase both synthetic and analyte targets react with additional amplifiers 53, 56 to produce both synthetic target/amplifier complex and an analyte target/amplifier complex. Exonuclease reaction then degrades the amplifiers to release both analyte 50 (two targets) and six distinct synthetic targets 53, 56. It can be seen that each phase results in a doubling of released target sequences, so that the amplification is likewise exponential. Table 2 shows the results of five phases or steps of this method.

TABLE 2
Dual target

| | | Derived Fragments | | |
|---|---|---|---|---|
| | original | | | N (s) |
| Step (s) | targets | sA | sB | total |
| initial | 2 | 0 | 0 | 0 |
| 1 | 2 | 1 | 1 | 2 |
| 2 | 2 | 3 | 3 | 6 |
| 3 | 2 | 7 | 7 | 14 |
| 4 | 2 | 15 | 15 | 30 |
| 5 | 2 | 31 | 31 | 62 |

FIG. 3C is a plot showing the data of Tables 1 and 2, depicting the increase or amplification of synthetic targets by the single target scheme and dual target scheme.

FIGS. 4A through 4B illustrate alternative apparatus and methods for reporter amplification and purification.

FIGS. 4A shows generally the elements illustrated in FIG. 2H, with modified reporter probes 61,62. In this example, the reporter probes are not bound to a substantial substrate, and have a capping ground 78 on the 3′ terminal end of the reporter oligonucleotide 35. Two alternatives are shown, one in which the capping group is present at the terminus of the complementary reporter sequence (35″) and one in which the capping group is present at the terminus of short tail sequence. Capping groups can be any of a number of suitable species known in the art which resist exonuclease attachment and/or processing, so as to protect the 3′ end from degradation, e.g., nucleotide analogs, covalently bound species and the like. Such capping groups can conveniently be attached during the course of conventional oligonucleotide synthesis. The modified reporter probes may be employed for analyte triggered reporter release in a solution phase.

FIGS. 4B shown an alternative example of a method and apparatus having aspects of the invention for purifying released reporters 35′ and 35″ prior to nanoelectronic detection, so as to simplify the detection environment and improve sensitivity and selectivity (e.g., “noise reduction”). The removal of high molecular weight polynucleotides prior to detector operation may be advantageous. The substrate may advantageously comprise a magnetic bead or one of the other alternative substrates describe herein. One or a plurality of “sandwich” probes 79 comprising an oligonucleotide having a sequence complementary to at least a portion of reporter 35′ or 35″ is bound to substrate 32 as described with respect FIGS. 1A-H, such as by a biotin/streptavidin bond. The reporter 35′ is shown hybridized by the short tail region to probe 79. The reporter 35′ is shown hybridized by at least a portion of the reporter sequence which is complementary to the nanocode of probes 60,61.

As described herein, the substrate-bound reporter 35′, 35″ may be rinsed or washed to remove the enzymatic reaction media with unbound species, and replaced by a buffer, which may be optimized for nanoelectronic detector operation (for example, by magnetic immobilization of beads). The probe 79 may have a nucleotide sequence selected to permit stable reporter attachment during washing, and to permit subsequent convenient denaturization to release reporters for detection.

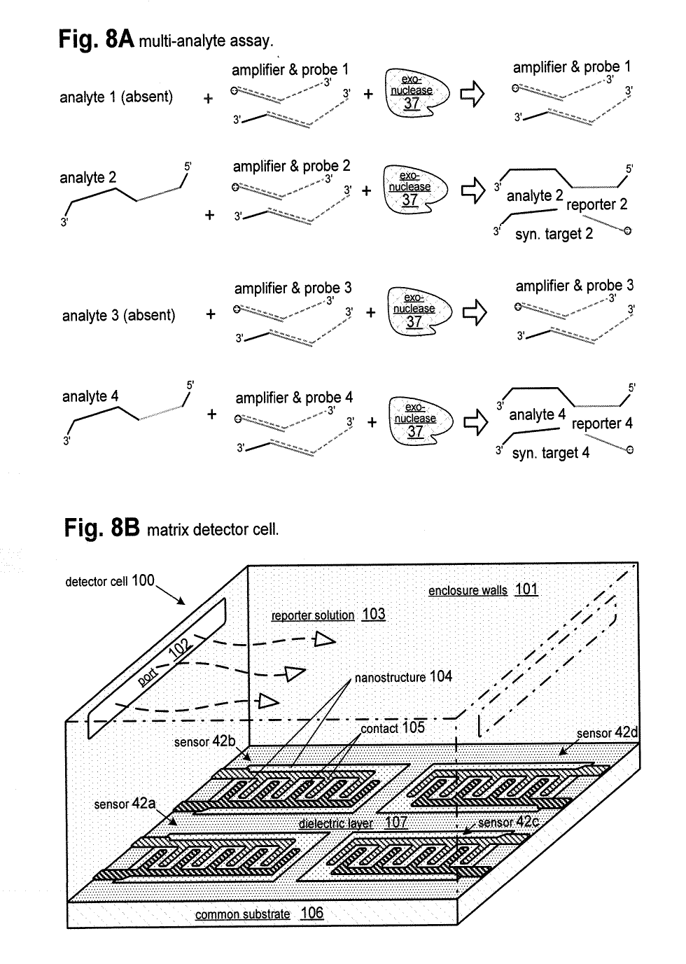

Note the multi-analyte assay and matrix detector embodiment described below with respect to FIGS. 8A-B. The reporter purification method shown in FIG. 4B may conveniently employ “sandwich” probes 79 complementary to a common sequence portion of the reporter 35, where other portions of the reporter sequence are specific to one of a plurality of analyte types. What ever reporter types are released in the assay reaction may then be collectively purified prior to matrix detector contact.

Exponential Non-PCR Target Amplification, Integrated Reporter Amplification

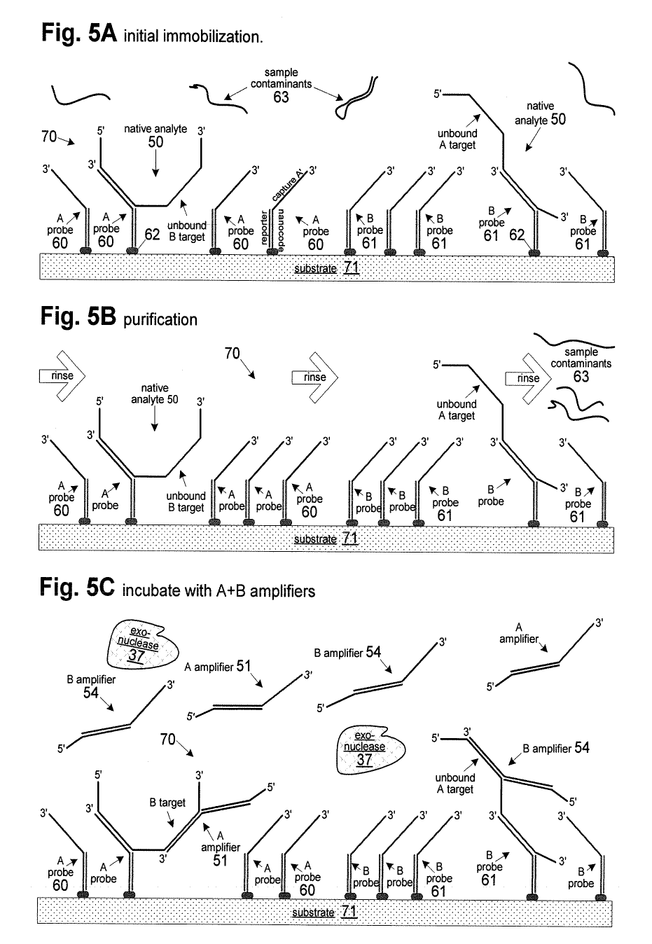

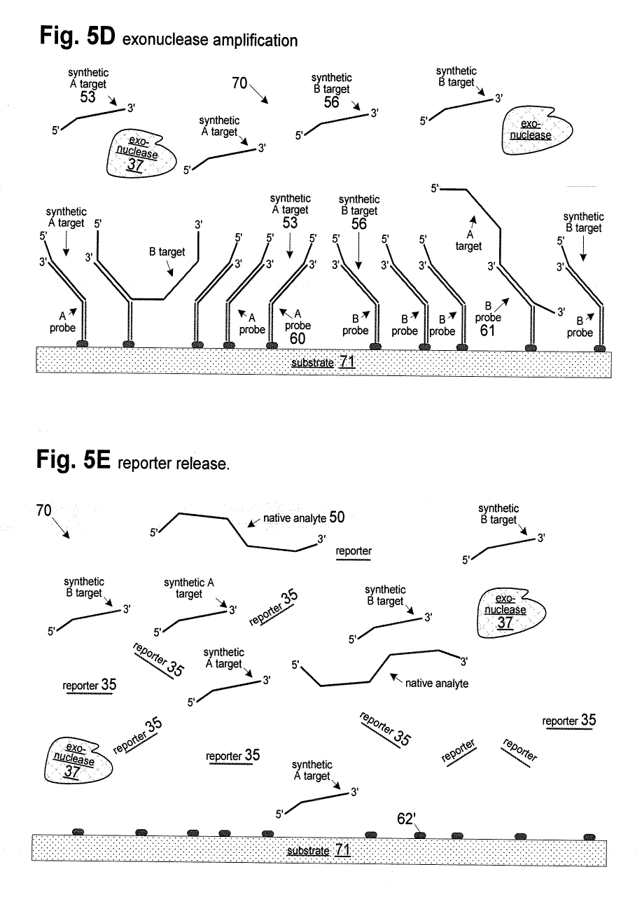

FIGS. 5A through 5E illustrate one exemplary method and apparatus embodiment having aspects of the invention (among a number of alternative methods having aspects in common) which provide exponential analyte-responsive amplification including a pre-amplification purification process, having a number of aspects in common with the methods and apparatus shown in FIGS. 2A-2H, and share common reference numerals in many cases. Methods and apparatus such as depicted in FIGS. 5A-5E may be employed independently or in combination with other methods described herein, in particular with the method and apparatus, such as is exemplified by FIGS. 1A-1H.

Initial immobilization. FIG. 5A shows the sample medium including a target analyte polynucleotide 50 comprising a target sequence A (target A), and also a target sequence B (target B), and including a variety of sample contaminants 63 (e.g., non-analyte polynucleotides, interfering enzymes, undesired chemical constituents, excess diluent, and the like). The sample is contacted, under conditions sufficient to effect polynucleotide hybridization, to an analysis system 70 comprising a substrate 71 and a plurality of capture probes 60, 61 attached to the substrate, for example by biotin-streptavidin bonding 62.

The probes comprise “A” probe 60 and “B” probe 61 (which may be essentially the same as probe 60, 61 in FIGS. 2A-H). The A probe 60 includes capture sequence A′ which is complementary to target sequence A of analyte polynucleotide 50, and the B probe 61 includes capture sequence B′ which is complementary to target sequence B of analyte polynucleotide 50. Preferably, particularly where the sample comprises a very small quantity of analyte species 50, system 70 comprises a sufficient plurality of A probes 60 and/or B probe 61 to bind a substantial fraction of the molecules of analyte 50 that may be present in the sample, so as to maximize sensitivity to rarified samples. In the schematic illustration of FIG. 2A, all analyte molecules 50 are bound, either to a probe 60 or a probe 61. Excess probes 60, 61 are functional for steps described below. 1. excess of A and B reporter probes bind large fraction of anaylyte molecules. 2. spacial separation of probe types (e.g., bead segregation, fixed substrates, etc.) avoids aglutination/cross-linking while leaving about half of the analyte A and target sites unhybridized.

Substrate 71 may be particulate (e.g., one or a plurality of beads) or non-particulate (e.g., a well, plate, belt or the like). In certain embodiments, substrate 71 is configured so as to reduce potential for multiply bound analyte polynucleotides 50 (an analyte hybridized at both A target and B target sequences to corresponding probes 60 and 61. In the example shown this is represented by separate right and left hand zones providing distinct regions for the one or more of A probe 60 and B probe 61, but many alternatives are possible. For example, alternative arrangements may have distinct bead types (e.g., optionally employing bead segregation mechanisms), fixed plate or lumen zones, variable stringency conditions, distinct melting/denaturation properties, and the like. In certain embodiments substrate 71 may be integral with nano electronic detector elements (described further below) or may be disposed separate from the detector.

Purification. FIG. 5B shows the elements of FIG. 5A, in which a rinsing and/or washing step is carried out to remove excess sample medium and contaminants 63, retaining the analyte 50 bound to substrates by probes 60, 61 (immobilization/separation techniques are used to retain any particulate substrates, if present).

Incubation. FIG. 5C shows the elements of FIG. 5B, in which the sample medium has been replace with a buffer including exonuclease 37 (e.g., having activity as in FIGS. 2A-H) and amplifier duplex oligonucleotides (may be essentially the same as “A” amplifier 51 and “B” amplifier 54 in FIG. 2A).

Amplification. FIG. 5D shows the elements of FIG. 5C, in which (under conditions suitable for hybridization and enzymatic activity) the A amplifier 51, B amplifier 54 and exonuclease 37 have reacted with analyte 50 (as well as subsequent synthetic targets) as depicted in FIG. 2B-2G, so as to release a plurality of synthetic A targets 53 and synthetic B targets 56 into the buffer medium.

In the example shown, synthetic A targets 53 and synthetic B targets 56 hybridize with excess of the plurality of pristine A probes 60 and B probes 61 provided in the step of FIG. 5A (alternatively, additional probes may be provided in the step of FIG. 5D). This reaction has produced a plurality of synthetic target/probe complexes in the manner depicted in FIG. 2H. In certain embodiments, an excess of amplifiers 51, 54 are provided to permit exonuclease-mediated amplification to continue so as to produce sufficient synthetic A and B targets to saturate a plurality of probes 60 and 61 respectively via hybrid complex formation.

Reporter release. FIG. 5E shows the elements of FIG. 5D, in which (under conditions suitable for enzymatic activity) exonuclease 37 has reacted with the synthetic target/probe complexes as well as the native analyte/probe complexes in the manner depicted in FIG. 2H, so as to degrade the probes 60 and 61 and release of reporter oligonucleotides 35 (as well as target species) into the buffer medium.

In the example shown, the exonuclease degrades all probes to release reporters for detection. The only substantial population of polynucleotides present are the native analyte and those species released in responsive to analyte presence (synthetic targets and reporters), and thus cross reactivity is minimized or avoided. Reporters can be optimized (e.g., in size or composition) for detector response. Note that the reaction of FIG. 5E may generally proceed simultaneously with the reaction shown in FIG. 5D, where the conditions permit. Alternatively, stringency controls, probe segregation, and the like may be used to isolate these reaction steps.

Note that the reporters 35 may be detected in the manner described with respect to FIGS. 1G-H (either via flow to an isolated nanoelectronic detector 40 or an integrated nanoelectronic detector communicating with the reaction region).

In certain alternative embodiments, substrate 71 may comprise one or more nanoelectronic detection elements, configured to directly detect the degradation of the probes 60, 61. For example, substrate 71 may comprise one or more regions of a nanoparticle, such as carbon nanotube elements communicating with electrical contacts. For example, changes in the properties one or more nanotubes due to probe degradation are detectable as described herein, and as described in Examples A-I of the incorporated patent applications.

In certain alternative embodiments, substrate 71 comprises one or more regions comprising an interlocking network of nanoparticles, such as carbon nanotubes, contacted by one or more electrodes, (e.g., at least one spaced-apart pair of source/drain electrodes). The nanoparticle network may be supported by wafer-like substrate structure (e.g., silicon, SiO2, Si3N4, PET, counter electrode material, and the like or combinations of these). The applications incorporated by reference herein provide a number of examples of the preparation and operation of such devices. The degradation of probes 60, 61 (in some cases leaving residual ligand moieties 62′) produces at least one change in the electrical, mechanical and/or electrochemical environment of the nanoparticle elements which is detectable by suitable circuitry (not shown), such as a change in capacitance of a nanoparticle relative to a counter electrode, a change in transistor characteristics under the influence of a gate electrode, and the like properties.

Amplification With Subsequent Enzyme Removal.

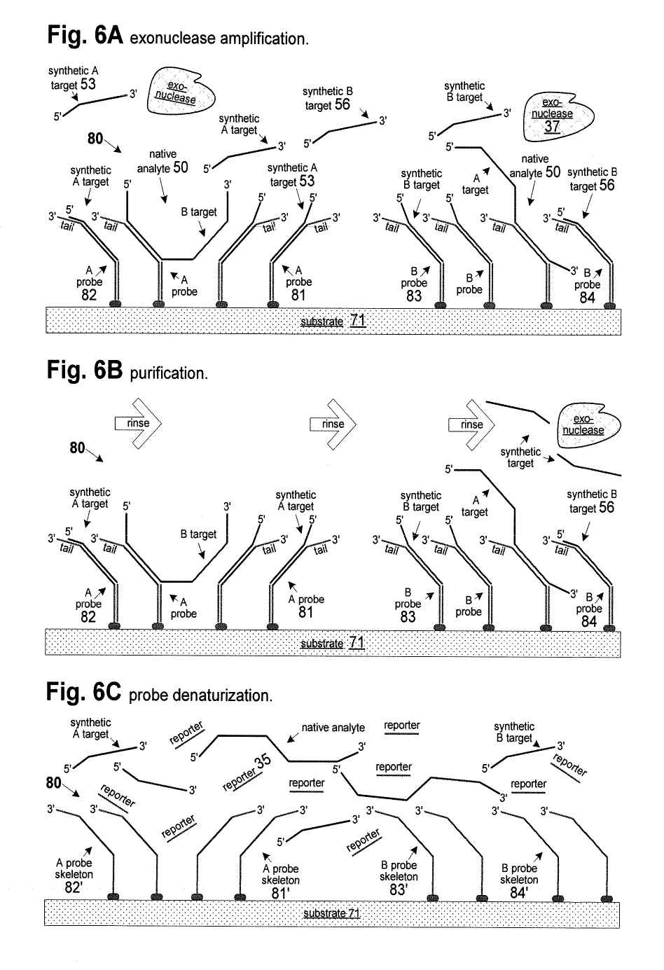

FIGS. 6A through 6C illustrate one exemplary method and apparatus embodiment having aspects of the invention (among a number of alternative methods having aspects in common) which provide exponential analyte-responsive amplification followed by removal of exonuclease from the media under analysis. The method and apparatus illustrated have a number of aspects in common with the methods and apparatus shown in FIGS. 2A-H and 5A-E, and share common reference numerals in many cases.

Exonuclease amplification. FIG. 6A shows the sample medium including a target analyte polynucleotide 50 comprising a target sequence A (target A), and also a target sequence B (target B). The sample may be processed in a manner generally the same or similar to the steps depicted in FIGS. 5A-D, so as to react analyte 50 (under conditions suitable for hybridization and enzymatic activity) with the A amplifier 51, B amplifier 54 and exonuclease 37, so as to release a plurality of synthetic A targets 53 and synthetic B targets 56 into the buffer medium.

In this alternative embodiment having aspects of the invention, exonuclease-mediated amplification is carried out so as to contact one or more modified A probes 81 and/or 82, and one or more modified B probes 83 and/or 84. The modified probes have protective 3′ tails which eliminate the non-protruding (e.g. blunt or recessed) 3′ end, so as to prevent degradation by the 3′ duplex-specific activity of exonuclease 37. In this manner the reaction of FIG. 5E is prevented. In the example shown, excess amplifier has permitted excess synthetic A and B targets to saturate the modified probes 81-84.

In first alternative form, modified A probe 81 and modified B probe 83 have protective 3′ tails which are non-complementary with an adjacent portion of the bound synthetic A and B target respectively, so that (in the probe/target hybrid complex) the protective tail has a single stranded 3′ terminal portion.

In a second alternative form, modified A probe 82 and modified B probe 84 have protective 3′ tails which are complementary with an adjacent portion of the bound synthetic A and B target respectively, so that (in the probe/target hybrid complex) the protective tail has a duplex or double-stranded 3′ terminal portion, the 3′ terminal portion protruding beyond the adjacent 5′ terminal end of the respective synthetic target oligonucleotide (sticky 3′ end).

Purification. FIG. 6B shows the elements of FIG. 6A, in which a rinse and/or wash step is carried out following probe/target complex formation (e.g., rinse exonuclease buffer from substrate-bound probe/target complex with a replacement buffer), so as to remove excess reagent material (and any remaining sample material) including exonuclease 37 from the medium. The replaced medium may be optimized for detector operation (and need not be suitable for enzymatic activity) as in the step illustrated in FIG. 1G.

Probe denaturization. FIG. 6C shows the substrate 71 and probe/target elements of FIG. 6B, in which denaturization step has been carried out to denature the probe/target complex (e.g., by use of heat, electric field, stringency controls) so as to release reporter molecules 35 and/or synthetic target molecules 53, 56. Note that depending on stringency controls and oligonucleotide sequence design, the denaturization process may be either total or selective. Thus a detector may be optimized to detect reporter 35, target 53, target 56, or any combination of these (alternative probes may optionally omit reporter 35).

One or more probe skeleton species 81′, 82′ 83′ and/or 84′ may remain attached to substrate 71 (in the example shown theses are single stranded oligonucletides). As in the example of FIG. 5E, the substrate 71 may comprise a nanoelectronic detector such as is described herein (direct substrate device detection), configured to detect the denaturization status directly. For example, direct substrate device detection may include detecting the difference between the denaturization event (e.g. comparison of signals before, during and after denaturization) involving pristine probes 81-84 (no analyte 50 present in sample) and the denaturization event involving probes/target complexes (created in response to the presence of analyte 50). Similarly, in additional alternatives, both such direct substrate device detection combined with remote nanodetector (e.g. as in FIG. 1H) may be carried out.

Reporter Probes With Internal Nuclease-resistant Blocking Groups.

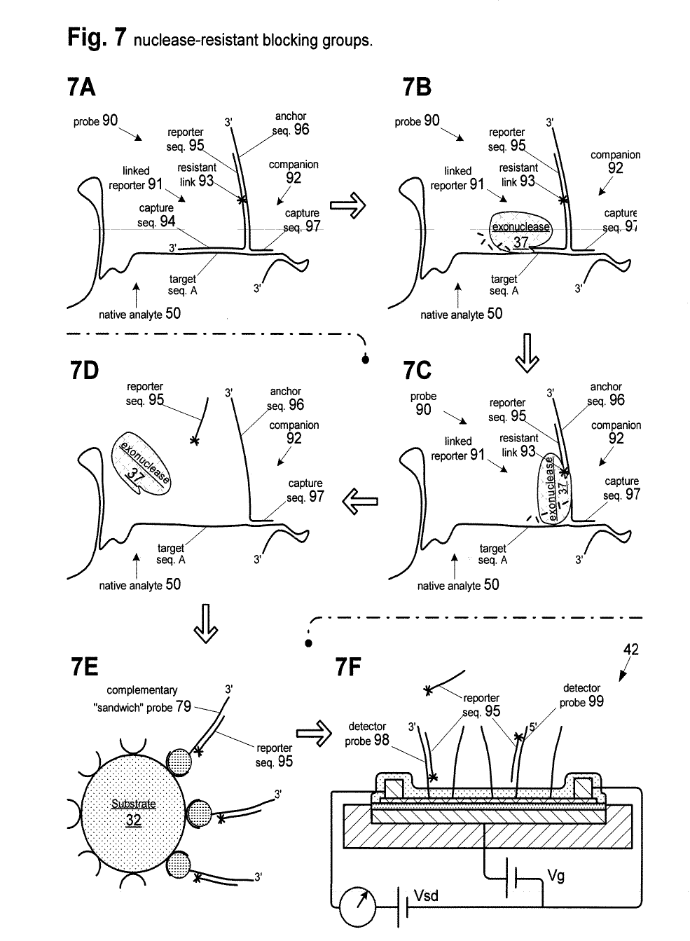

FIGS. 7A-F shows an example of a reporter probes having aspects of the invention and including with internal blocking groups which stop the processing of exonuclease at the sequence location of the blocking group. Such blocking groups are known in the art, and may include, for example, analogs to natural nucleic acids, covalently bonded species.

Hybridize probes. FIG. 7A shows an analyte polynucleotide 50 which has been treated with a probe 90 so as to hybridize probe 90 to a target sequence A of the analyte 50 so as to form an analyte/probe complex. The probe 90 includes a linked reporter oligonucleotide 91 and a companion oligonucleotide 92.

The linked reporter oligonucleotide 91 includes a proximal capture sequence portion 94 complementary to at least a portion of target A of analyte 50, and includes a distal reporter sequence portion 95 complementary to at least a portion of companion oligonucleotide 92 The capture sequence 94 and reporter sequence 95 are linked by an intervening resistant link group 93 (proximal and distal in this example are arbitrarily described with 3′ as proximal, 5′ as distal, it being understood that alternative embodiments may have 5′ exonuclease activity and a “mirror image” oligonucleotide structure with reversal of the 3′ vs. 5′ sense).

The companion oligonucleotide 92 includes a proximal anchor sequence 96 complementary to at least a portion of the reporter sequence 95, and a distal capture sequence 97 complementary to at least a portion of analyte 50.

In certain embodiments, an assembled probe 90 including a hybridized duplex of linked reporter 91 and companion 92 may be provided, e.g., in a reagent solution, and reacted with a sample including analyte 50. In alternative embodiments, a single-stranded linked reporter 91 and a single stranded companion 92 may be provided, and each contacted with analyte 50, so as to hybridize in situ with each other and with analyte 50, forming an analyte/probe complex as shown in FIG. 7A.

Degrade probes. FIG. 7B shows the elements of FIG. 7, further including a exonuclease 37 having activity to degrade duplex polynucleotide with non-protruding 3′ end. The exonuclease has attached to and begun degrading the proximal portion of linked reporter 91 comprising capture sequence 94.

FIG. 7C shows the elements of FIG. 7B, the exonuclease 37 having continued to degrade linked reporter 91 so as to remove the portion of linked reporter 91 proximal to resistant link 93.

Release reporter. FIG. 7D shows the elements of FIG. 7C, the exonuclease 37 having detached form the probe analyte complex upon reaching resistant link 93, reporter seq. 35 remaining intact. Reporter seq. 95 is shown denatured and detached form companion 92. Preferably, either or both of the conditions of the reaction medium (temperature, pH, ionic composition, and the like), and the length and nucleotide composition of reporter seq. 35 and the corresponding complementary portion of companion anchor sequence 96 may be selected so that the duplex of the remaining reporter seq. 95 is generally unstable (as duplex form) when the proximal portion of oligonucleotide 91 is degraded. Alternatively, conditions may be controlled following enzymatic degradation to promote denaturization of this reporter 95 duplex.

Following detachment of reporter 95, in certain embodiments, companion 92 is configured to remain attached to analyte 50. In alternative embodiments, companion 92 may also be denatured and detach from analyte 50. In either case, the companion 92 may be recycled by binding with additional pristine linked reporter oligonucleotide 91 in the media, so as to form an additional probe/analyte complex, as shown in FIG. 7A.

Purification. FIG. 7E shows an optional purification step in which one or more complementary “sandwich” probes 79 are provided, having one or more probe oligonucleotides attached to a substrate, each having a sequence complementary to reporter sequence 95, e.g., in the manner described with respect to FIG. 4B. Binding of reporter 95 to probe 79, followed by rinsing/washing of substrate 32, enables the sample medium to be highly simplified upon release (via denaturization) of reporter 95.

Detection. FIG. 7F illustrates the detection of reporters 95 by a nanoelectronic sensor 42 having aspect so the invention, as described with respect to FIG. 1G-H. Note that as descibed above, reporters having aspects of the invention may be detected by a variety of alternative means, including convention optical methods and the like. Detector 42 has one or more detector probes 98 attached, with can hybridize via complementary sequences with reporter 95, so as to produce a detectable change in sensor properties. Removal or deactivation of exonuclease 37, such as by optional purification step of 7E, removes constraints on probe/reporter complex configuration in that a distal (with respect to sensor) non-protruding 3′ end is not degraded (rapid detection by sensor 42 also may also obviates this constraint). Alternatively probe 98 may be configured (as shown) to have a either a distal protruding 3′ end. Alternative detector probe 99 has a distal 5′ end. Note that in the example shown, resistant link 93 is at or adjacent to the 3′ end of reporter 95, so as to resist endonuclease activity in the duplex probe/reporter complex.

Multi-Analyte Assay With Multiple Reporter Types And A Matrix Detector Cell

FIGS. 8A and 8B illustrate one exemplary method and apparatus embodiment having aspects of the invention including a multi-analyte assay with multiple reporter types and a matrix detector cell. The method includes providing a plurality of probe types configured to amplify and provide detection species (e.g., reporters) corresponding to a plurality of different analytes.

FIG. 8A is a flow diagram depicting a multi-analyte assay with multiple reporter probe types, and optionally having purification steps and multiple amplifier types. In this example, a reagent mixture is compounded having both reporter probes and amplifiers corresponding to putative analytes 1, 2, 3 and 4 respectively. A sample medium containing only analytes 2 and 4 is contacted to the reagent mixture and exonuclease 37, under conditions effective to promote hybridization and enzyme activity. Following amplification and reporter release reactions as described above, reporter (as well as synthetic targets) are released corresponding to analytes 1 and 2. Reporter probes and amplifiers corresponding to analytes 1 and 3 remain unreacted. Note that the reporter probes and amplifiers (and rinsing or purification) may be any of the embodiments described herein, such as with respect to FIGS. 1-7, or similar embodiments without departing from the spirit of the invention.

FIG. 8B illustrates detector cell 100 having enclosure walls 101 and one or more ports 102, and a matrix of sensors, each having a distinct detector sensitivity corresponding to a different analyte reporter molecule (four are shown, sensors 42a, b, c and d). Note that the sensors may include any of the sensor embodiments described herein and in the incorporated patent applications. In this example, each sensor 42 includes a substrate (e.g., a common substrate 106 having a dielectric layer 107 is shown, such as a silicon wafer and SiO2 surface layer, alternatively a polymer sheet, or the like), a nanostructure element 104 disposed adjacent the substrate (in this example an interconnected CNT network) and at lease one contact 105 in electrical communication with the nanostructure element 104 (an interdigitated pair of source-drain electrodes are shown. In a transistor example, separate gate electrodes may be included, or alternatively, a substrate material such as a doped Si wafer may serve as a common gate electrode for the matrix. Nanostructure element 104 in each sensor is sensitized with a different recognition material specific to one of the putative analytes of the assay, in this example a oligonucleotide detector probe complementary to a selected one of the reporter oligonucleotides corresponding to one of analytes 1, 2, 3 and 4 respectively.

Detection solution 103, in this example a purified reporter solution derived form the reaction mixture of FIG. 8A, is contacted to the sensors 42 in cell 100. Suitable measurement circuitry (not shown) detects changes in the properties of those ones of sensor 42a-d in which hybridization with a corresponding reporter occurs.

Note that although in the matrix cell embodiment 100 shown, the sensors 42 are configured to detect a distinct reporter molecule, alternative embodiments are possible without departing from the spirit of the invention, such a having sensors including probes sensitive to synthetic targets or analyte molecules.

Aptamer-Reporter Complex.

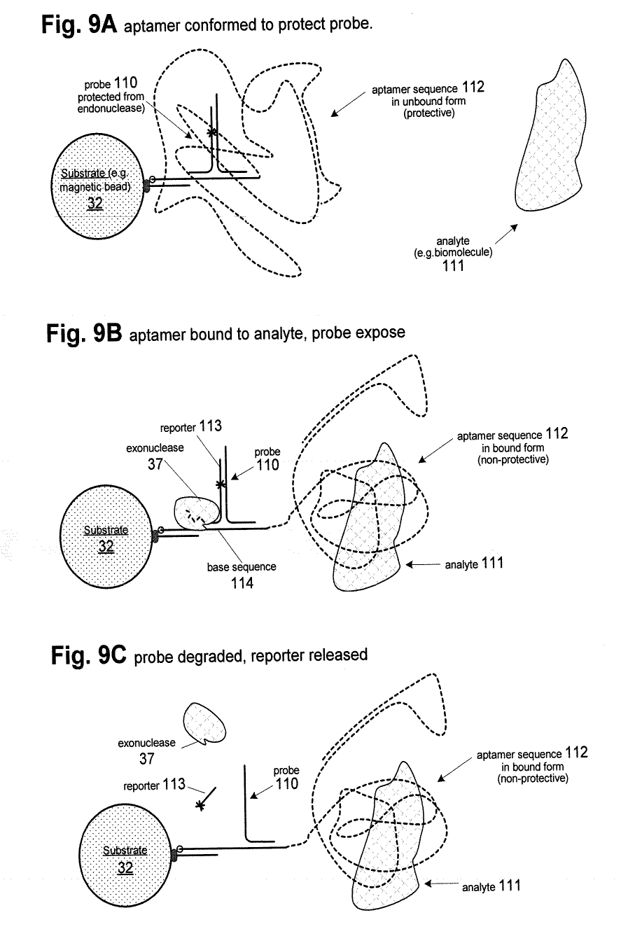

FIGS. 9A to 9C illustrate one exemplary method having aspects of the invention employing a probe assembly including an aptamer portions for detection of analytes, including biomolecules. The aptamer/base sequence composite is selected so that there is a conformal change upon binding of the aptamer 112 to analyte 111. In the example shown, an Aptamer-Reporter Complex is immobilized to a substrate, such as a bead, e.g., by methods described above. As shown in FIG. 9B, probe assembly 110 includes a reporter 113 in a duplex form bound to base sequence 114. The probe 110 can generally similar to any of the examples described herein. In the example shown, the probe 110 is generally similar to that described with respect to FIGS. 7A-F, in which the base sequence 114 acts in the same manner as the analyte 50. The base sequence is connected to an aptamer sequence 112, the aptamer having a specific binding capacity for a target analyte 111. In embodiments having aspects of the invention, the analyte may be any one of a number of substances having a specific affinity or reactivity with an aptamer or similar polynucleotide construct, e.g., a globular protein, a polysaccharide, or the like.

Aptamer conformed to protect probe. As shown in FIG. 9A, when the aptamer 112 is not bound to analyte 111, the conformation is protective of the probe 110, in that the endonuclease 37 is prevented from substantially degrading the probe.

Aptamer bound to analyte, probe expose. As shown in FIG. 9B, when the aptamer 112 is bound to analyte 111, the conformation exposes the probe 110 to the endonuclease 37, so that the probe 110 is attacked by endonuclease 37

Probe degraded, reporter released. As shown in FIG. 9C, subsequent degradation of probe 110 by enzyme 37 releases reporter 113, so it may be detected in the manner described above with respect to the embodiments of FIGS. 1-8.

### Nanoelectronic Sensors for Polynucleotide Detection

### EXAMPLES A THROUGH I

Additional exemplary embodiments having aspects of the invention are described in Examples A through I set forth in the co-invented U.S. applications Ser. No. 11/318,354 filed Dec. 23, 2005 (see WO2006-071,895) and Ser. No. 11/212,026 filed Aug. 24, 2005 (see WO2006-024,023), each of which applications is incorporated by reference.

Multiple-stage Amplifiers (Power-law Amplification).

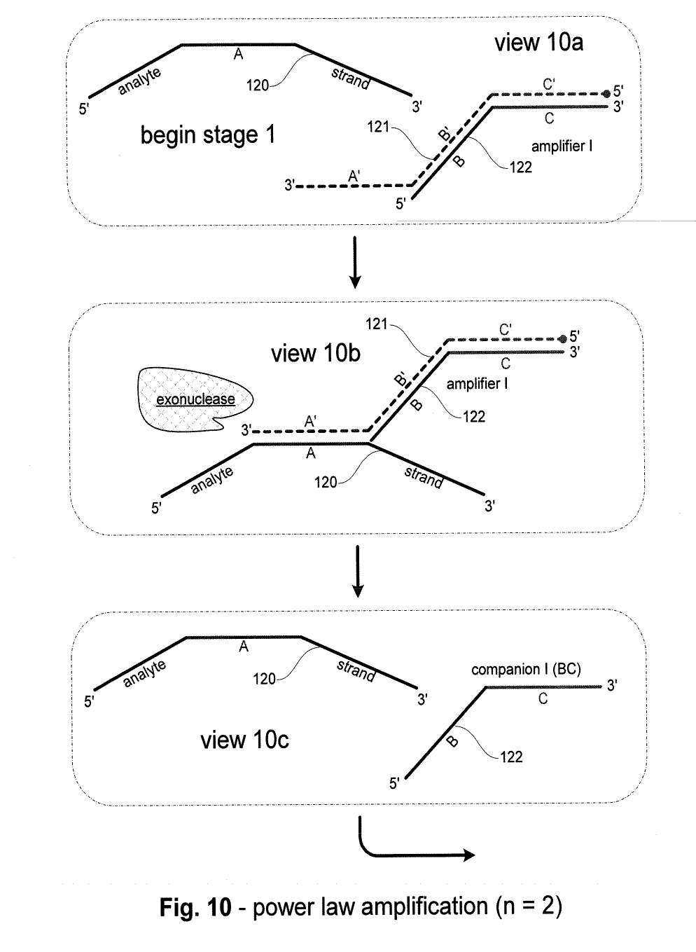

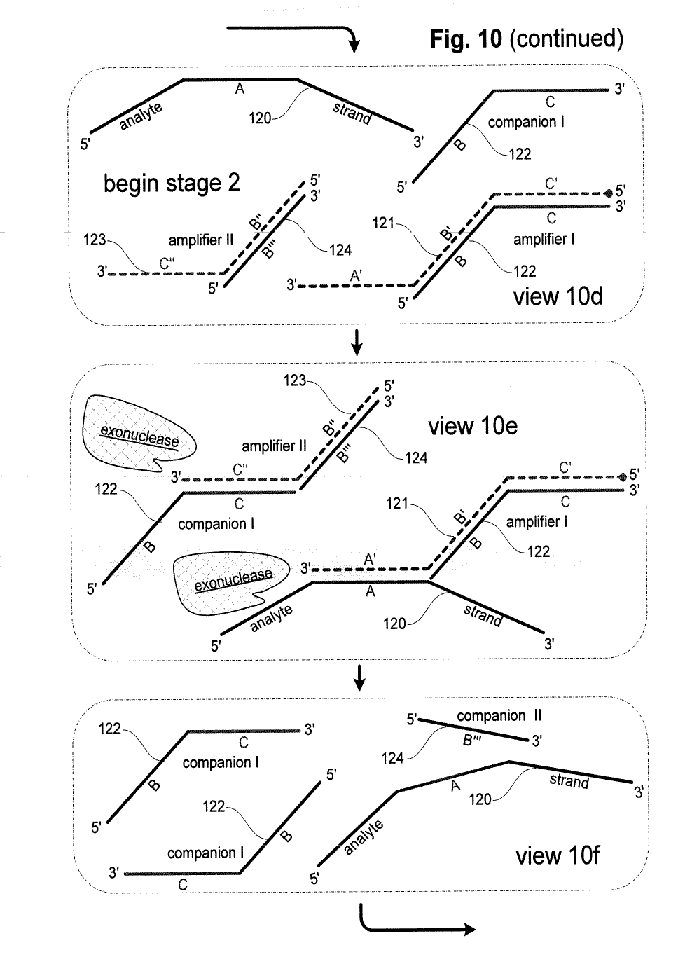

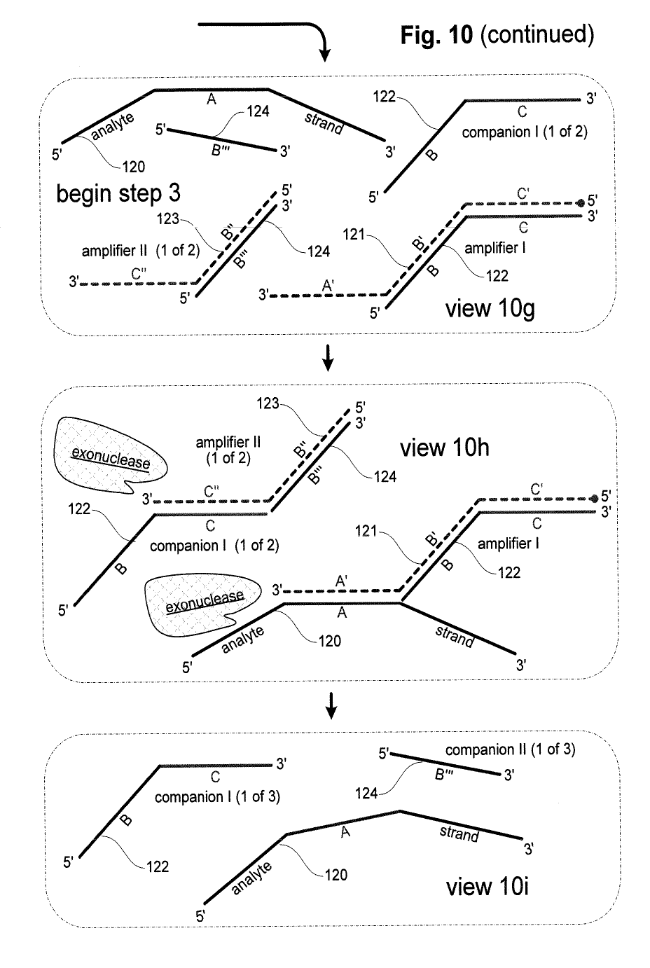

FIGS. 10-13 depict exemplary embodiments having aspects of the invention which provide for analyte-responsive enzyme-mediated amplification in a manner similar in a number of respects and operative schemes as the embodiments described above with respect to FIGS. 2 and 3, and wherein a multi-stage series of amplifier reagent species is employed.

In the embodiments of FIGS. 10-13, the product of the analyte-responsive enzyme reaction of an initial stage amplifier forms the substrate for the amplification of a second-stage amplifier, so as to produce a second amplification product upon subsequent enzyme action. Additional stages may be include.

In the examples, only the initial stage of amplification is triggered directly by analyte presence, via hybridization with a pre-selected analyte target sequence A. Subsequent stage amplifiers are triggered by amplification products. The accumulation of the products of amplification (derived strands or reporter fragments) tends to follow a power-law relationship based on the number of successive amplifier stages (n=2,3, . . . ).

Detector devices employing such multiple-stage amplification schemes may detect any or all of such amplification products (reporters), so as to permit measurement of the presence or concentration of a biomolecule analyte in a sample. Such detection may employ any of the nanoelectronic devices described herein.

For purposes of illustration of aspects of the invention, in the examples of FIGS. 10-13 the oligonucleotide complexes and enzymes are configured for degradation by a 3′-exonuclease selective for a initiation on a duplex DNA having a non-protruding 3′ terminus on one strand, with degradation proceeding in 3′ to 5′ direction along the non-protruding strand. However, as in the case of the examples discussed above with respect to FIGS. 2 and 3, other configurations are possible without departing from the spirit of the invention, and other enzyme types may be employed, such as a 5′ exonuclease, DNA polymerase, and the like, with amplifier reagents species configured accordingly.

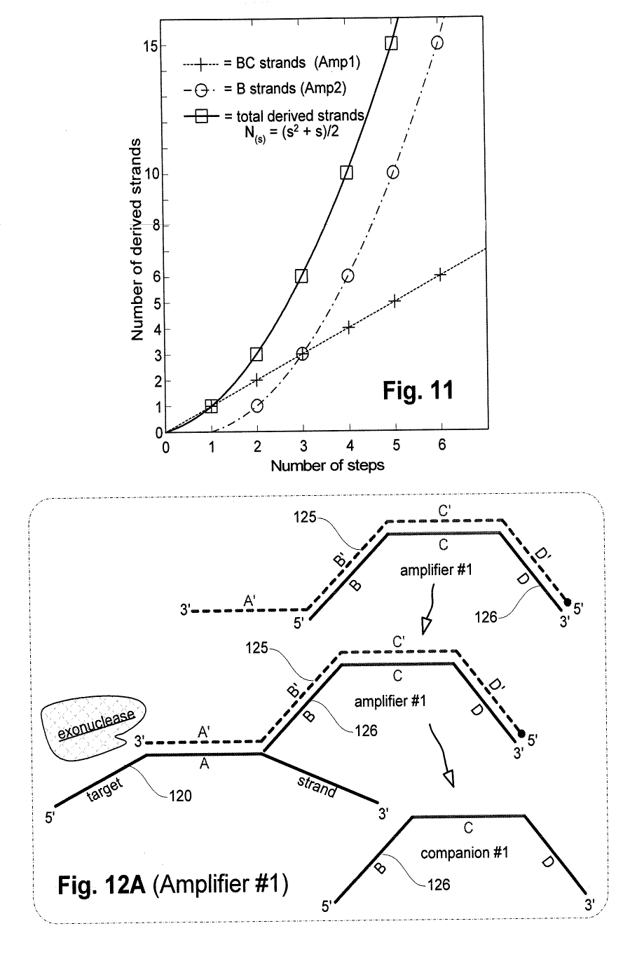

Two-stage amplification. For example, FIGS. 10 and 11 depict a two-stage (n=2) method embodiment. As in the other embodiments, an initial analyte-responsive enzyme-mediated reaction releases “synthetic” (i.e., pre-prepared/selected natural or synthesized oligonucleotides not present in sample) targets which participate in additional enzyme-mediated reactions which in turn result in the release of further synthetic strands or fragments. The synthetic oligonucleotides are provided in a duplex form configured to be protected from exonuclease activity in the absence of analyte polynucleotide in the sample.

Initial conditions. In the example illustrated in FIG. 10, the first view portion, view 10a, shows the initial state of sample measurement in which:
a. Analyte polynucleotide strand 120, having a pre-selected sequence A is exposed, under conditions sufficient to effect polynucleotide hybridization, to a reagent/buffer having at least Amplifier I;
b. Amplifier I comprises a duplex structure which includes a capture strand 121 and a companion strand 122;
c. Capture strand 121 comprises (in 3′ to 5′ order) a capture sequence A′ (complementary to all or a portion of target A), and two additional sequences B′ and C′; and
d. Companion strand 122 comprises (in 3′ to 5′ order) two companion sequences C and B, which are complementary to and hybridized to (all or a portion of) the companion sequences C′ and B′ respectively of capture strand 121, so as to leave the capture sequence A′ of strand 121 exposed.

Stage 1. In view 10b, Amplifier I is shown in duplex association with analyte strand 120 by means of hybridization of the capture sequence A′ of strand 121 to target sequence A of analyte strand 120. An exonuclease species (together with any necessary co-factors) is added or present in the reagent, so as to initiate degradation of capture strand 121 at its 3′ terminus.

In view 10c, enzymatic degradation of stage 1 is illustrated as complete, with the undegraded analyte strand 120 (original strand) and companion strand 122 (companion I or BC) released into solution. At this point (end of step 1) there is one original strand and one derived (e.g., reporter) strand in solution.

Stage 2. View 10d illustrates the beginning of stage 2 of the method, in which the products of stage 1 (stands 120 and 122) are shown exposed in the reagent/buffer medium to additional Amplifier I and also to Amplifier II:
a. Amplifier II comprises a duplex structure includes a capture strand 123 and a companion strand 122;
b. Capture strand 123 comprises (in 3′ to 5′ order) a capture sequence C″ (complementary to all or a portion of sequence C of companion strand 122), and additional sequence B″; and
c. Companion strand 124 comprises companion sequence B′″, which is complementary to and hybridized to (all or a portion of) the companion sequence B″ of capture strand 123, so as to leave the capture sequence C″ of strand 123 exposed.

In view 10e, Amplifier II is shown in duplex association with companion strand 122 by means of hybridization of the capture sequence C″ of strand 123 to sequence C of companion strand 122. An exonuclease species (together with any necessary co-factors) is added or present in the reagent, so as to initiate degradation of capture strand 123 at its 3′ terminus. Simultaneously, additional Amplifier I is shown in duplex association with analyte strand 120 as described for stage 1.

In view 10f, enzymatic degradation of stage 2 is illustrated as complete, with the undegraded analyte strand 120 (original strand), two of companion strand 122 (first derived and second derived); and companion strand 124 (companion II or B) released into solution. At this point (end of step 2) there is one original strand and 3 derived (e.g., amplicon or reporter) strands in solution.

Additional steps (phases 1 and 2). View 10g illustrates the beginning of subsequent step 3 of the method, in which the products of both stage 1 (strands 120 and 122) and stage 2 (stand 124) are shown exposed in the reagent/buffer medium to additional Amplifier I and Amplifier II:

In view 10h, Amplifier I is shown in duplex association with analyte strand 120 (stage 1) and Amplifier II is shown in duplex association with companion strand 122 (stage 2). Exonuclease initiates degradation of capture strand 121 of the analyte duplex and capture strand 123 of the each of the companion I duplexes.

In view 10i, enzymatic degradation is illustrated as complete, with the undegraded analyte strand 120, three of companion strand 122; and three of companion strand 124 (one from step 2 and two newly derived) released into solution. At this point (end of step 3) there is one original strand and 6 derived (e.g., amplicon or reporter) strands in solution.

Table 3 shows the results of six phases or steps of this method.

TABLE 3
(n = 2)

| | | derived fragments | | |
|---|---|---|---|---|
| | original | BC | B | N (s) |
| Step (s) | target | (amp 1) | (amp 2) | total |
| initial | 1 | 0 | 0 | 0 |
| 1 | 1 | 1 | 0 | 1 |
| 2 | 1 | 2 | 1 | 3 |
| 3 | 1 | 3 | 3 | 6 |
| 4 | 1 | 4 | 6 | 10 |
| 5 | 1 | 5 | 10 | 15 |
| 6 | 1 | 6 | 15 | 21 |

FIG. 11 is a plot which shows the data of table 3 with respect to a two-stage amplification.

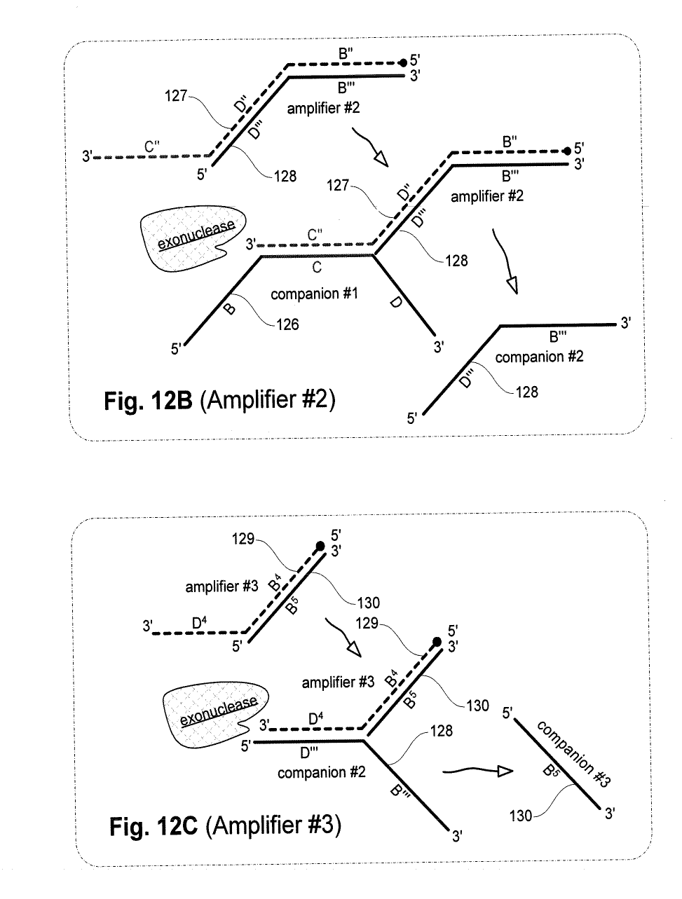

Three-stage amplification. In an alternative example, FIGS. 12A-C and 13 depict a three-stage (n=3) method embodiment, similar in many respect to the two-stage example shown in FIGS. 11-12. For convenience, only the mode of operation of each amplifier stage is described in detail below, the underlying principles and configuration being clear when read in conjunction with the description above. Note that, as in the previous example, each stage may proceed simultaneously provided the particular precursor target, and sufficient amplifier reagent and enzyme are present.

Stage 1. FIGS. 12A shows three portions connected by arrows depicting the progressive activity of Amplifier #1, in which:
a. Amplifier #1 comprises a duplex structure which includes a capture strand 125 and a companion strand 126;
b. Capture strand 125 comprises (in 3′ to 5′ order) a capture sequence A′ (complementary to all or a portion of target A), and three additional sequences B′, C′ and D′;
c. Companion strand 126 comprises (in 3′ to 5′ order) three companion sequences D, C and B, which are complementary to and hybridized to (all or a portion of) the companion sequences D′, C′ and B′ respectively of capture strand 125, so as to leave the capture sequence A′ of strand 125 exposed;
d. Analyte polynucleotide strand 120, having a pre-selected sequence A, is exposed, under conditions sufficient to effect polynucleotide hybridization, to a reagent/buffer having at least Amplifier #1, so as to bind and produce a duplex Analyte-Amplifier #1 complex;
e. An exonuclease species (together with any necessary co-factors) is added or present in the reagent, so as to initiate degradation of capture strand 125 at its 3′ terminus; and
f. enzymatic degradation continues to completion, so as to release the undegraded analyte strand 120 and companion strand 126 (companion #1 or BCD) into solution.

Stage 2. FIGS. 12B shows three portions connected by arrows depicting the progressive activity of Amplifier #2, in which
a. Amplifier #2 comprises a duplex structure which includes a capture strand 127 and a companion strand 128;
b. Capture strand 127 comprises (in 3′ to 5′ order) a capture sequence C″ (complementary to all or a portion of sequence C of strand 126), and two additional sequences D″ and B″;
c. Companion strand 128 comprises (in 3′ to 5′ order) two companion sequences B′″ and D′″, which are complementary to and hybridized to (all or a portion of) the companion sequences B″ and D″ respectively of capture strand 127, so as to leave the capture sequence C″ of strand 127 exposed;
d. Companion #1 (strand 126) is exposed, under conditions sufficient to effect polynucleotide hybridization, to a reagent/buffer having at least Amplifier #2, so as to bind and produce a duplex Companion #1-Amplifier #2 complex;
e. An exonuclease species (together with any necessary co-factors) is added or present in the reagent, so as to initiate degradation of capture strand 127 at its 3′ terminus; and
f. enzymatic degradation continues to completion, so as to release the undegraded Companion #1 (126) and companion strand 128 (companion #2 or BD) into solution.

Stage 3. FIGS. 12C shows three portions connected by arrows depicting the progressive activity of Amplifier #3, in which
a. Amplifier #3 comprises a duplex structure which includes a capture strand 129 and a companion strand 130;
b. Capture strand 129 comprises (in 3′ to 5′ order) a capture sequence D⁴ (complementary to all or a portion of sequence C′″ of strand 128), and an additional sequence B⁴;
c. Companion strand 130 comprises a companion sequence B⁵, which is complementary to and hybridized to (all or a portion of) the companion sequence B⁴ of capture strand 129, so as to leave the capture sequence D⁴ of strand 129 exposed;
d. Companion #2 (strand 128) is exposed, under conditions sufficient to effect polynucleotide hybridization, to a reagent/buffer having at least Amplifier #3, so as to bind and produce a duplex Companion #2-Amplifier #3 complex;
e. An exonuclease species (together with any necessary co-factors) is added or present in the reagent, so as to initiate degradation of capture strand 129 at its 3′ terminus; and
f. enzymatic degradation continues to completion, so as to release the undegraded Companion #2 (126) and companion strand 130 (companion #2 or B) into solution.

Table 4 shows the results of six phases or steps of this method.

TABLE 4
(n = 3)

| | | derived fragments | | | |
|---|---|---|---|---|---|
| | original | BCD | BD | B | N (s) |
| Step (s) | target | (amp 1) | (amp 2) | (amp 3) | total |
| initial | 1 | 0 | 0 | 0 | 0 |
| 1 | 1 | 1 | 0 | 0 | 1 |
| 2 | 1 | 2 | 1 | 0 | 3 |
| 3 | 1 | 3 | 3 | 1 | 7 |
| 4 | 1 | 4 | 6 | 4 | 14 |
| 5 | 1 | 5 | 10 | 10 | 25 |
| 6 | 1 | 6 | 15 | 20 | 41 |

FIG. 13 is a plot which shows the data of table 4 with respect to a three-stage amplification.

Homologous sequences. With respect to the examples herein, and in particular the multi-stage examples of FIGS. 10-13, it should be understood that the separate designation of homologous sequences which correspond to a sequences present in more than one amplifier reagent species, does not imply that such homologous sequences are identical in nucleotide length or sequence, or in overall chemical composition. Such homologous sequences may be identical, or they may differ in a number of respects.

For example, in FIGS. 12B-12C, sequence D′″ of strand 128 may be seen to hybridize with sequence D″ (in Amplifier #2, strand 127) and also hybridize with sequence D⁴ (in Amplifier #3-Companion #2 complex, strand 129). Such homologous sequences may be configured to have an effective degree of complementary properties to carry out their respective hybridization functions in the inventive methods, without necessarily being identical in sequence, length, or incorporation of additional chemical groups. Thus, for example, sequences D″ and D⁴ may be configured to have different degrees of completeness of hybridization or may have different denaturalization or annealing stringency properties. Similarly, sequences D″ and D⁴ may be configured to have different lengths, terminal groups, intermediate groups, non-natural nucleotides, or the like, e.g., so to regulate the initiation, termination or specificity or blocking of enzymatic activity, as the case may be.

In other alternatives, homologous sequences in amplicon or reporter species (e.g., B′″ in strand 128 and B5 in strand 130) may have different labels, markers or other detection-enhancing properties, e.g., so as to permit distinct and separate detection (for example in quantitative amplification tracking), or detection by different devices or methods. In certain embodiments, multiple and/or quantitative detection schemes may be employed, e.g., to reduce false positives, to distinguish between similar analytes, and the like.

### Example

### Single-target Amplification With Fluorescent Detection

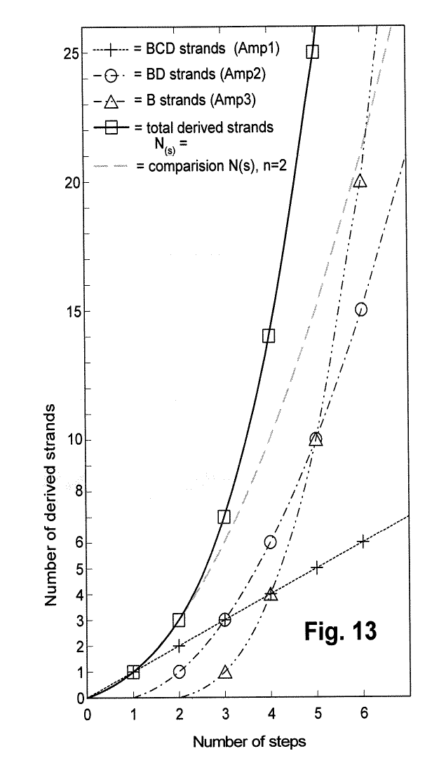

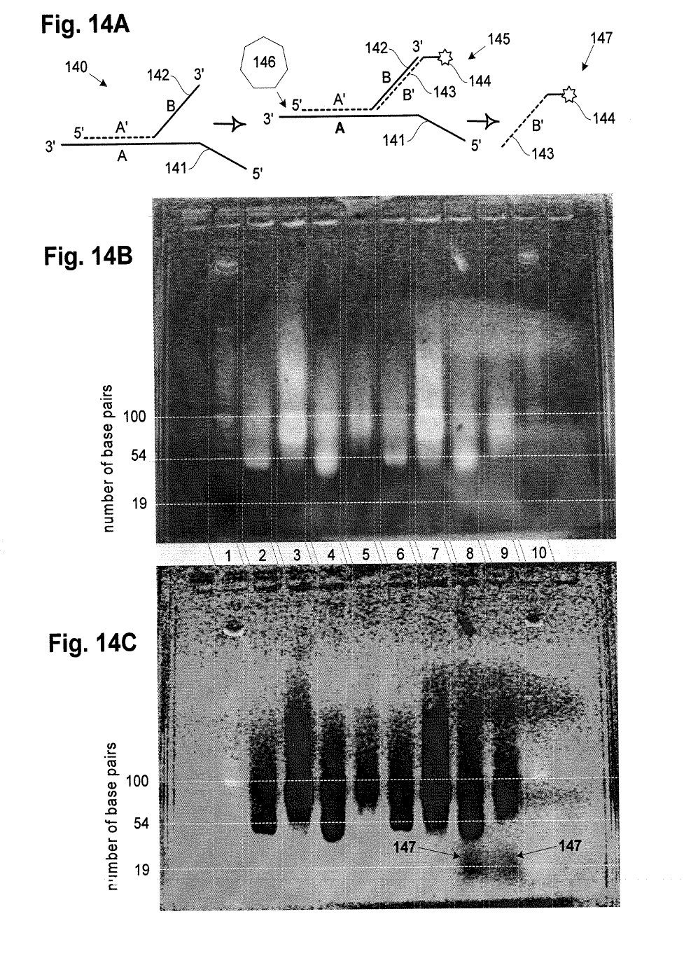

FIGS. 14A-14C illustrate an example of single-target amplification method having aspects of the invention (See, e.g., FIG. 3A) utilizing fluorescent detection, through one step of the method.

In this example, the amplifier capture probe sequence is configured to form an analyte-amplifier complex whereby the capture stand has a terminal non-protruding 5′ end, subject to degradation by a 5′>>3′ exonuclease. The enzyme used in this example is a T7 polymerase having 5′>>3′ exonuclease activity.

As described above several alternative embodiments of amplification and detection methods having aspect of the invention may be practiced employing a variety of nucleotide-active enzymes, including 3′-exonuclease, 5′-exonuclease, DNA polymerase (e.g., via proof-reading or nuclease activity), and the like. For example, T7 DNA polymerase has been demonstrated using methods of the invention. Similarly, alternative amplifier systems having aspects of the invention may utilize the activity of endonucleases when configured to form an analyte-amplifier complex with a suitable enzyme initiation site, without departing from the spirit of the invention.

FIG. 14A is a diagram showing the components of the analyte and amplifier system 140, including analyte 141 having a target sequence A (in this example, having about 54 bases), capture strand 142 (in this example, having about 102 bases) and having a complementary sequence A′ and a extended sequence B, and reporter strand 147 (in this example, having about 19 bases). The of amplifier-analyte complex 145 in this example has about 121 base pairs.

Reporter strand 147 includes strand 143 which has a complementary sequence B′ which binds to extended sequence B′ of strand 142, and in this example includes also a FAM fluorescent group 144, attached to strand 143 by conventional practice, to permit convenient detection by optical methods.

FIGS. 14B-14C show the results of electorphoretic gel separation of various components of the reactions of the method, where FIG. 14B is a positive photograph of a electrophoretic gel following separation, and where FIG. 14C is a negative version of the photograph of FIG. 14B, which provides a more distinct indication of the data in a photo-reproducible gray-scale image suitable for patent illustration. In this illustration of the method, various combinations of reaction or reagent components were tested for comparison purposes that need not necessarily be present in a useful reagent system. The gel was configured to have 10 parallel channels, in which the buffer in each channel is described in the list below:
1—Oligomer calibration ladder of units of about 10 base pairs (bp), the “bright line” component corresponding to 100 bp.
2—analyte oligomer 141 (˜54 bp).
3—analyte 141 hybridized to capture sequence 142 (˜102 bp).
4—analyte 141 hybridized to sequence 142, with low enzyme concentration.
5—analyte 141 hybridized to sequence 142, with high enzyme concentration.
6—analyte oligomer 141.
7—amplifier-analyte complex 145 (˜121 bp).
8—amplifier-analyte complex 145, with low enzyme concentration.
9—amplifier-analyte complex 145, with high enzyme concentration.
10—oligomer calibration ladder

As may be seen from FIGS. 14B-C, the enzymatic digestion product, reporter 147, is not visible in channels 7 corresponding to the complex without enzyme, but is readily apparent at the 19-mer level in both channels 8 and 9, corresponding to the enzymatic digestion reaction mixture. One of ordinary skill in the art will readily appreciate that the concentration of enzyme and other reaction conditions may be optimized without undue experimentation of facilitate convenient, sensitive and selective detection of such an analyte.

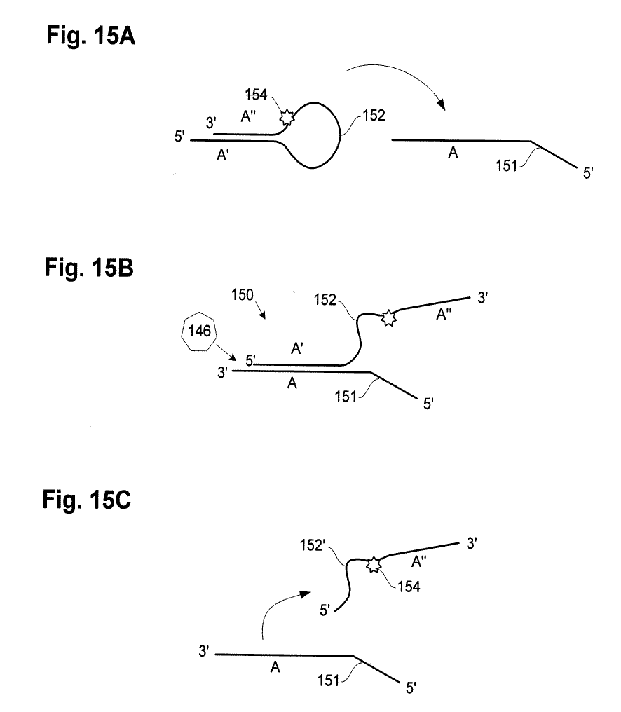

Hairpin probe assembly. Further exemplary embodiments having aspects of the invention may eliminate separate capture and reporter portions of the probe assembly. FIGS. 15A-15C illustrate an example of a “hairpin” type probe assembly. In this example, the probe assembly comprises a co-linear polynucleotide strand 152 which includes a capture portion A′ (complementary to the target A of analyte strand 151) and a reporter portion, which may have one or more labels or other detection enhancement 154 (e.g., a fluorescent marker group) disposed on or adjacent any convenient place on the strand 152. In the example a marker 154 is shown in a mid-portion of stand 152, but it may be disposed alternatively, such as adjacent the 3′ end.

As shown in FIG. 15A, strand 152 includes at least one addition portion A″ which has a degree of hybridization affinity such that portions A′ and A″ tend to self-hybridize (e.g., to form a hairpin-like configuration) when not in association with the target portion A of the template species 151 so as to be protected from degradation by the selected enzyme. In this example the hairpin form of strand 152 has a protruding 5′ end, thus being protected from an enzyme 146 (e.g. an endonuclease) requiring a duplex having a non-protruding 5′ terminus for initiation of activity.

As shown in FIG. 15B, when the probe strand 152 is associated with target strand 151, the affinity of A′ for the target A displaces the equilibrium to favor formation of a complex 150, in which the strand 152 is associated with strand 151 so as to have a non-protruding 5′ terminus, which is attacked by enzyme 146.

As shown in FIG. 15C, enzymatic degradation continues until shortened strand 152′ is released. Target 151 is then available for further amplification. In a multistage method, released strand 152′ may serve as a template for suitably configured amplifiers.

Having thus described a preferred embodiment and methods, it should be apparent to those skilled in the art that certain advantages have been achieved. It should also be appreciated that various modifications, adaptations, and alternative embodiments thereof may be made within the scope and spirit of the present invention.

1. A method of introducing a reporter species into a medium where the medium includes a biomolecular template species, the method comprising a non-PCR, template-triggered, enzyme-activated release of the reporter species from a probe assembly having a binding affinity for the template species, the method including in any operative order the steps of:
(a) providing at least a first probe assembly, the first probe assembly including:
(i) at least a first probe strand having a capture nucleotide sequence which provides a selective binding affinity for a target portion of the template species;
(ii) at least one first reporter species including a binding portion having a polynucleotide sequence configured to hybridize with a corresponding binding portion of the probe strand;
(iii) wherein the first probe assembly includes at least one first probe strand and at least one first reporter species hybridized to comprise a polynucleotide duplex probe assembly; and
(iv) wherein the duplex probe assembly is configured so as to have at least one enzyme-initiation site suited to promote the action of a selected enzyme having nuclease activity sufficient to degrade all or a portion of the probe strand so as to release the first reporter species from the probe assembly, the enzyme-initiation site being formed in the event that the capture nucleotide sequence binds with all or part of the selected target portion of the template species so as to form a template-probe complex;
(b) contacting the medium with at least one first probe assembly under conditions effective to promote binding of the selected target portion of the template species to the capture nucleotide sequence portion of the probe polynucleotide to form a first template-probe complex having an enzyme-initiation site;
(c) contacting the medium with at least the selected enzyme under conditions effective to promote nuclease activity of the enzyme at an enzyme-initiation site, so that in the event that a first template-probe complex has been formed in step (b), the first probe strand is degraded so as to release the first reporter species from the probe assembly.

2. The method of claim 1, wherein the biomolecular template species includes an analyte species in a sample, and wherein the first reporter species is configured to be directly or indirectly detectable when released from the probe assembly, the method further including the steps of:
(d) directly or indirectly detecting the released first reporter species;
(e) determining at least a presence or concentration of the analyte species in the sample based on the direct or indirect detection of the first reporter species.

3. The method of claim 2, wherein the first reporter species is configured to be directly detectable when released from the first probe assembly.

4. The method of claim 3, wherein the first reporter species includes at least a detection portion including at least one of a detectable polynucleotide sequence or a detectable label group.

5. The method of claim 2, wherein the first reporter species is configured to be indirectly detectable when released from the first probe assembly.

6. The method of claim 5, wherein the first reporter species includes a template portion which is configured to act as a target template for at least one second non-PCR, template-triggered, enzyme-activated release of a second reporter species from a second probe assembly having a binding affinity for the template portion of the first reporter species.

7. The method of claim 6, wherein the second reporter species is configured to be directly detectable when released from the second probe assembly.

8. The method of claim 6, wherein the first reporter species is configured to be indirectly detectable when released from the first probe assembly; and wherein the second reporter species includes a template portion which is configured to act as a target template for at least one third non-PCR, template-triggered, enzyme-activated release of a third reporter species from a third probe assembly having a binding affinity for the template portion of the second reporter species.

9. The method of claim 1, wherein the biomolecular template species includes at least a non-polynucleotide portion, wherein the capture nucleotide sequence of the first probe assembly includes an aptamer, and the selective binding affinity includes a binding interaction of the aptamer with at least the non-polynucleotide portion.

10. The method of claim 1, wherein the biomolecular template species includes at least a polynucleotide portion, and wherein the capture nucleotide sequence of the first probe strand provides a selective binding affinity for a target portion of the template species by complementary nucleotide hybridization.

11. The method of claim 1, wherein the first probe strand and the first reporter species comprise a co-linear polynucleotide strand, wherein portions of the co-linear polynucleotide strand are configured to self-hybridize when not in association with the target portion of the template species so as to be protected from degradation by the selected enzyme.

12. A method of detecting an analyte polynucleotide in a sample, the method comprising:
(a) providing at least a first probe assembly, the first probe assembly including:
(i) at least a first polynucleotide probe strand having a capture nucleotide sequence which provides a selective hybridization affinity for a selected target nucleotide portion of the analyte;
(ii) at least one first reporter strand including a binding portion having a nucleotide sequence configured to hybridize with a corresponding binding portion of the probe strand;
(iii) wherein the first probe assembly includes at least one first probe strand and at least one first reporter hybridized to comprise a polynucleotide duplex probe assembly; and
(iv) wherein the duplex probe assembly is configured so as to have at least one enzyme-initiation site suited to promote the action of a selected enzyme having nuclease activity sufficient to degrade all or a portion of the probe strand so as to release the first reporter strand from the probe assembly, the enzyme-initiation site being formed in the event that the capture nucleotide sequence hybridizes with all or part of the target nucleotide portion of an analyte to form a duplex analyte-probe complex;
(b) contacting the sample with at least one first probe assembly under conditions effective to promote binding of the target nucleotide portion to the capture nucleotide sequence portion of the probe polynucleotide to form a first analyte-probe complex having an enzyme-initiation site;
(c) contacting the sample with at least the selected enzyme under conditions effective to promote nuclease activity of the enzyme at an enzyme-initiation site, so that in the event that a a first analyte-probe complex has been formed in step (b), the first probe strand is degraded so as to release the first reporter strand from the probe assembly;
(d) directly or indirectly detecting the released reporter strand;
(e) determining at least a presence or concentration of the analyte based on the direct or indirect detection of the reporter strand.

13. A method of detecting an analyte polynucleotide in a sample, the method comprising:
(a) providing at least a first probe assembly, the first probe assembly comprising:
(i) a substrate;
(ii) at least one probe polynucleotide including a proximal 5′-terminal nucleotide and a distal 3′-terminal nucleotide, the probe polynucleotide bound adjacent the proximal 5′-terminal nucleotide to the substrate, the probe polynucleotide comprising at least one nanocode nucleotide sequence portion, and a capture nucleotide sequence portion having a nucleotide sequence complementary to a corresponding target nucleotide sequence of the analyte polynucleotide;
(iii) at least a first reporter polynucleotide having a nucleotide sequence complementary to the nanocode sequence of the probe polynucleotide, the first reporter polynucleotide hybridized to the nanocode sequence to form a duplex portion with the probe polynucleotide;
(iv) the probe polynucleotide so configured that the capture nucleotide sequence portion and 3′-terminal nucleotide extends distally from the first reporter polynucleotide duplex portion;
(b) contacting the first probe assembly with a sample which putatively contains the analyte polynucleotide, under conditions effective to allow for binding of the target nucleotide sequence of the analyte polynucleotide to the capture nucleotide sequence portion of the probe polynucleotide to form a duplex first probe/analyte complex, the first probe/analyte complex including:
(i) the first probe assembly;
(ii) at least one analyte polynucleotide;
(iii) the analyte polynucleotide extending distally to form either of (1) a blunt distal 5′-terminal end or (2) a protruding distal 5′-terminal end;
(c) contacting the first probe/analyte complex with an exonuclease, the exonuclease having 3′-to-5′ exo-deoxyribonuclease activity including specific binding to double-stranded DNA followed by selective hydrolysis of the 3′ terminated strand of the DNA duplex;
(d) maintaining conditions effective to allow binding of the exonuclease to the first probe/analyte complex and effective to allow hydrolysis of the distally 3′ terminated strand of the first probe/analyte complex by the exonuclease to an extent that the analyte polynucleotide and at least the first reporter polynucleotide are released from the first duplex probe/analyte complex; and
(e) determining the presence of the analyte polynucleotide in the sample by detecting the presence of at least the first reporter polynucleotide released by the exonuclease hydrolysis.

14. The method of claim 13, further comprising:
(f) providing at least a second probe assembly comprising:
(i) a substrate; wherein the substrate is either of (1) the same substrate as the first probe assembly or (2) a substrate distinct from the substrate of the first probe assembly;
(ii) at least one probe polynucleotide including a proximal 5′-terminal nucleotide and a distal 3′-terminal nucleotide, the probe polynucleotide bound adjacent the proximal 5′-terminal nucleotide to the substrate, the probe polynucleotide comprising at least one nanocode nucleotide sequence portion, and a capture nucleotide sequence portion having a nucleotide sequence complementary to a corresponding target nucleotide sequence of the analyte polynucleotide;
(iii) at least a second reporter polynucleotide having a nucleotide sequence complementary to the nanocode sequence of the probe polynucleotide, the second reporter polynucleotide hybridized to the nanocode sequence to form a duplex portion with the probe polynucleotide;
(iv) the probe polynucleotide so configured that the capture nucleotide sequence portion and 3′-terminal nucleotide extends distally from the second reporter polynucleotide duplex portion;
(g) following the release of the analyte polynucleotide in step (d) contacting the second probe assembly with a sample containing the analyte polynucleotide released in step (d) under conditions effective to allow for binding of the target nucleotide sequence of the released analyte polynucleotide to the capture nucleotide sequence portion of the probe polynucleotide of the second probe assembly to form a second duplex probe/analyte complex;
(h) contacting the second probe/analyte complex with an exonuclease, the exonuclease having 3′-to-5′ exo-deoxyribonuclease activity including specific binding to double-stranded DNA followed by selective hydrolysis of the 3′ terminated strand of the DNA duplex;
(i) maintaining conditions effective to allow binding of the exonuclease to the second duplex probe/analyte complex and effective to allow hydrolysis of the distally 3′ terminated strand of the probe/analyte complex by the exonuclease to an extent that the analyte polynucleotide and at least the second reporter polynucleotide are released from the second duplex probe/analyte complex; and
(j) wherein step (e) includes determining the presence of the analyte polynucleotide in the sample by detecting the presence of at least both the first reporter polynucleotide released from the first duplex probe/analyte complex and at least the second reporter polynucleotide released from the second duplex probe/analyte complex.

15. The method of claim 14, further comprising:
(k) repeating steps (f) through (i) of claim 2 until at least about an order of magnitude increase in the presence of released reporter polynucleotide is obtained relative to the presence of released reporter polynucleotide following step (d), so as to comprise an amplified released reporter polynucleotide presence.
(l) wherein step (e) includes determining the presence of the analyte polynucleotide in the sample by detecting the amplified released reporter polynucleotide presence.

16. The method of claim 14, wherein the method is the mirror-image method relative to claim 13 in which 3′ and 5′ terminal ends are reversed throughout the definition of the claim, so that:
(a) the substrate is conjugated adjacent a proximal 3′ end of the probe polynucleotide;
(b) the distal end of the first probe/analyte complex includes the analyte polynucleotide extending distally to form either of (1) a blunt distal 3′-terminal end or (2) a protruding distal 3′-terminal end;
(c) the exonuclease has 5′-to-3′ exo-deoxyribonuclease activity including specific binding to double-stranded DNA followed by selective hydrolysis of the 5′ terminated strand of the DNA duplex;
(d) the method includes maintaining conditions effective to allow hydrolysis of the distally 5′ terminated strand of the probe/analyte complex to release the analyte polynucleotide and at least the first reporter polynucleotide; and
(e) the method includes determining the presence of the analyte polynucleotide in the sample by detecting the presence of the reporter polynucleotide released by the exonuclease 5′-to-3′ hydrolysis.

17. The method of claim 13, wherein the detection of the reporter polynucleotide of step (e) is carried out by operation of an electronic sensor system comprising:
(a) at least one sensor platform having at least one electrical property, the platform including:
(i) a substrate,
(ii) at least an electrode disposed adjacent the substrate, and
(iii) at least one nanostructured element disposed adjacent the substrate, the nanostructured element in electrical communication the electrode;
(iv) at least one detector probe operatively associated with the nanostructured element, the probe including a detector polynucleotide having at least one nucleotide sequence which is complementary to a corresponding nucleotide sequence of the reporter polynucleotide and configured to bind to at least the first reporter polynucleotide; and
(b) electronic measurement circuitry connected to the at least one electrode and configured to measure a change in the at least one electrical property of the sensor platform due to binding of the detector probe with at least the reporter polynucleotide.

18. The method of claim 13, wherein the, further comprising following forming the first probe/analyte complex in step (b):
(m) separating the sample from the first probe/analyte complex; and
(n) carrying out subsequent steps in one or more media which are distinct from the sample.

19. The method of claim 18, further comprising following separating the sample in step (n):
(o) rinsing the first probe/analyte complex prior to step (p).

20. The method of claim 18, wherein providing at least a first probe assembly in step (a) includes providing plurality of probe assemblies such that:
(i) the substrate of each probe assembly is either of (1) a substrate in common with another probe assembly of the plurality of probe assemblies, or (2) a substrate distinct from the substrates of the other probe assemblies of the plurality of probe assemblies;
(ii) the plurality of probe assemblies is sufficient so that a substantial fraction of the analyte polynucleotide molecules present in the sample are bound in carrying out step (b) to form a corresponding plurality of probe/analyte complexes

21. The method of claim 18, wherein more than one of the plurality of probe assemblies comprise a common substrate.

22. The method of claim 18, wherein the substrate comprises a magnetic bead, and wherein separating the sample from the first probe/analyte complex in step (m) includes magnetically influencing the bead.

23. The method of claim 18, wherein the substrate comprises a particle including electrically charged species, and wherein separating the sample from the first probe/analyte complex in step (m) includes electrophoretically influencing the particle.

24. The method of claim 18, wherein the substrate comprises a particle having a selected size, and wherein separating the sample from the first probe/analyte complex in step (m) includes filtration of the particle from the sample.

25. The method of claim 18, wherein the substrate comprises surface, and wherein separating the sample from the first probe/analyte complex in step (m) includes fluid flow of the sample relative to the surface.

26. The method of claim 25, wherein the surface comprises at least a portion of a microfluidic enclosure.

27. The method of claim 13, wherein the first probe assembly includes more than one reporter polynucleotide, each of which is released during step (d).

28. The method of claim 13, further comprising, prior to step (c), treating the sample so as to inactivate or remove exonuclease present in the sample.

29. A method of detecting and distinguishing a plurality of analyte polynucleotide species in a sample, the method comprising:
(a) providing a first probe assembly as defined in claim 13, the first probe assembly comprising a first type of reporter polynucleotide and a capture nucleotide sequence complementary to a target nucleotide sequence specific to a first analyte polynucleotide species;
(b) providing at least a second probe assembly as defined claim 1, the second probe assembly comprising a second type of reporter polynucleotide and a capture nucleotide sequence complementary to a target nucleotide sequence specific to a second analyte polynucleotide species;
(c) carrying out at least steps (b) through (d) of claim 1, so as to release the first type of reporter polynucleotide in response to presence of the first analyte polynucleotide species in the sample, if the first analyte be present, and so as to release the second type of reporter polynucleotide in response to presence of the second analyte polynucleotide species in the sample, if the second analyte be present;
(d) determining the presence of the first analyte polynucleotide in the sample by detecting the presence of the first type of reporter polynucleotide released by the exonuclease hydrolysis; and
(e) determining the presence of the second analyte polynucleotide in the sample by detecting the presence of the second type of reporter polynucleotide released by the exonuclease hydrolysis distinct from the detection of the first type of reporter polynucleotide in step (d).

30. The method of claim 29, wherein the detection of reporter polynucleotides in steps (d) and (e) are carried out by operation of an electronic sensor system comprising:
a first sensor platform having at least one electrical property, the platform including:
(i) a substrate,
(ii) at least an electrode disposed adjacent the substrate, and
(iii) at least one nanostructured element disposed adjacent the substrate, the nanostructured element in electrical communication the electrode;
(iv) at least one detector probe operatively associated with the nanostructured element, the probe including a first type of detector polynucleotide having at least one nucleotide sequence which is complementary to a corresponding nucleotide sequence of the first type of reporter polynucleotide and configured to bind to at least the first type of reporter polynucleotide; and
at least a second sensor platform having at least one electrical property, the platform including:
(i) a substrate, wherein the substrate is either of (1) the common substrate with the first sensor platform, or (2) a substrate distinct from the substrate of the first sensor platform;
(ii) at least an electrode disposed adjacent the substrate, and
(iii) at least one nanostructured element disposed adjacent the substrate, the nanostructured element in electrical communication the electrode;
(iv) at least one detector probe operatively associated with the nanostructured element, the probe including a first type of detector polynucleotide having at least one nucleotide sequence which is complementary to a corresponding nucleotide sequence of the first type of reporter polynucleotide and configured to bind to at least the first type of reporter polynucleotide; and
electronic measurement circuitry connected to the at least one electrode of the first and second sensor platforms, and configured
(i) to carry out at least a first measurement of a change in the at least one electrical property of the first sensor platform due to binding of the detector probe with at least the first type of reporter polynucleotide; and
(ii) to carry out at least a second measurement, distinct from the first measurement, of a change in the at least one electrical property of the second sensor platform due to binding of the detector probe with at least the second type of reporter polynucleotide.

31. A method of detecting and distinguishing a plurality of analyte polynucleotide species in a sample, the method comprising:
(a) providing a first probe assembly as defined in claim 16, the first probe assembly comprising a first type of reporter polynucleotide and a capture nucleotide sequence complementary to a target nucleotide sequence specific to a first analyte polynucleotide species;
(b) providing at least a second probe assembly as defined claim 4, the second probe assembly comprising a second type of reporter polynucleotide and a capture nucleotide sequence complementary to a target nucleotide sequence specific to a second analyte polynucleotide species;
(c) carrying out at least steps (b) through (d) of claim 4, so as to release the first type of reporter polynucleotide in response to the presence of the first analyte polynucleotide species in the sample and so as to release the second type of reporter polynucleotide in response to the presence of the second analyte polynucleotide species in the sample;
(d) determining the presence of the first analyte polynucleotide in the sample by detecting the presence of the first type of reporter polynucleotide released by the exonuclease hydrolysis; and
(e) determining the presence of the second analyte polynucleotide in the sample by detecting the presence of the second type of reporter polynucleotide released by the exonuclease hydrolysis distinct from the detection of the first type of reporter polynucleotide in step (d).

32. The method of claim 31, wherein the detection of reporter polynucleotides in steps (d) and (e) are carried out by operation of an electronic sensor system comprising:
a first sensor platform having at least one electrical property, the platform including:
(i) a substrate,
(ii) at least an electrode disposed adjacent the substrate, and
(iii) at least one nanostructured element disposed adjacent the substrate, the nanostructured element in electrical communication the electrode;
(iv) at least one detector probe operatively associated with the nanostructured element, the probe including a first type of detector polynucleotide having at least one nucleotide sequence which is complementary to a corresponding nucleotide sequence of the first type of reporter polynucleotide and configured to bind to at least the first type of reporter polynucleotide; and
at least a second sensor platform having at least one electrical property, the platform including:
(i) a substrate, wherein the substrate is either of (1) the common substrate with the first sensor platform, or (2) a substrate distinct from the substrate of the first sensor platform;
(ii) at least an electrode disposed adjacent the substrate, and
(iii) at least one nanostructured element disposed adjacent the substrate, the nanostructured element in electrical communication the electrode;
(iv) at least one detector probe operatively associated with the nanostructured element, the probe including a first type of detector polynucleotide having at least one nucleotide sequence which is complementary to a corresponding nucleotide sequence of the first type of reporter polynucleotide and configured to bind to at least the first type of reporter polynucleotide; and
electronic measurement circuitry connected to the at least one electrode of the first and second sensor platforms, and configured
(i) to carry out at least a first measurement of a change in the at least one electrical property of the first sensor platform due to binding of the detector probe with at least the first type of reporter polynucleotide; and
(ii) to carry out at least a second measurement, distinct from the first measurement, of a change in the at least one electrical property of the second sensor platform due to binding of the detector probe with at least the second type of reporter polynucleotide.

33. A method of amplifying and detecting a representative target oligonucleotide species in response to the presence of an analyte polynucleotide in a sample, the method comprising:
(a) providing at least a first duplex oligonucleotide amplifier assembly configured to hybridize to a first target sequence portion of the analyte polynucleotide and comprising a first representative target oligonucleotide species;
(b) providing at least a second duplex oligonucleotide amplifier assembly configured to hybridize to a second target sequence portion of the analyte polynucleotide and comprising a second representative target oligonucleotide species;
(c) contacting the first and second amplifier assemblies to the analyte polynucleotide under conditions effective to promote sequence hybridization so as to hybridize at least a portion of the first and second amplifier assemblies to the first and second target sequence portions of the analyte polynucleotide, respectively, to form at least one amplifier/analyte complex;
(d) treating the at least one amplifier/analyte complex with a nuclease under conditions effective to promote nuclease activity, so as to release at least one of the first and second representative target oligonucleotide species from its corresponding amplifier assembly and from the amplifier/analyte complex; and
(e) detecting the presence of at least the released representative target oligonucleotide species so as to determine the presence of the analyte polynucleotide in a sample.

34. The method of claim 33, wherein:
in step (a), the first amplifier assembly comprises:
(i) at least a first amplifier polynucleotide including a proximal 5′-terminal nucleotide and a distal 3′-terminal nucleotide, the first amplifier polynucleotide comprising:
at least distal capture sequence portion (B′) which is complementary to a corresponding target sequence portion (B) of the analyte polynucleotide;
at least a proximal capture sequence portion (A′) which is distinct from capture sequence portion (B′) and which is complementary to a corresponding target sequence portion (A) of the analyte polynucleotide;
(ii) at least a first companion polynucleotide including a proximal 3′-terminal nucleotide and a distal 5′-terminal nucleotide, the first companion polynucleotide comprising at least a target sequence portion (A″) which is complementary to and hybridized with the proximal capture sequence portion (A′) of the first amplifier polynucleotide.
in step (b), the second amplifier assembly comprises:
(i) at least a second amplifier polynucleotide including a proximal 5′-terminal nucleotide and a distal 3′-terminal nucleotide, the second amplifier polynucleotide comprising:
at least a distal capture sequence portion which has substantially the sequence of the capture sequence portion (A′) of the first amplifier polynucleotide;
at least a proximal capture sequence portion which is distinct from the distal capture sequence portion and which has substantially the sequence of the capture sequence portion (B′) of the first amplifier polynucleotide;
(ii) at least a second companion polynucleotide including a proximal 3′-terminal nucleotide and a distal 5′-terminal nucleotide, the second companion polynucleotide comprising at least a target sequence portion (B″) which is complementary to and hybridized with the proximal capture sequence portion of the second amplifier polynucleotide.

35. An array system for detecting and distinguishing a plurality of analyte polynucleotide species in a sample, comprising:
(a) a reaction cell configured for the reaction of reporter probes and/or amplifiers with a sample putatively containing one or more analyte species;
(b) at least one purification mechanism (in the reaction cell or a separate cell), for purifying at least one of a reporter and/or a synthetic target;
(c) an array or matrix of nanoelectronic sensors (as described herein) each sensor including a recognition material (e.g., a detector probe comprising an oligonucleotide complementary oligonucleotide) sensitive to a reporter or synthetic target representative of a selected one the plurality of analytes);
(d) measurement circuitry configured to determine from signals derived for each sensor, which, if any, of the plurality of putative analytes are present in the sample.

36. A method of detecting analyte in a sample, the method comprising:
(a) providing a selected nuclease;
(b) providing at least a probe assembly comprising:
a capture oligonucleotide sequence configured to bind to a target portion of the analyte, and
a reporter oligonucleotide sequence representative of the analyte,
the reporter oligonucleotide sequence and capture oligonucleotide sequence provided in a duplex form having a double stranded portion;
the duplex form configured to be relatively resistant to degradation by the nuclease when presented to the nuclease in isolation; and
the duplex form configured to be relatively subject to degradation by the nuclease when presented to the nuclease in a form wherein the capture oligonucleotide sequence is bound to the target portion of the analyte to form an analyte/probe complex, the degradation by the nuclease sufficient to release at least the reporter oligonucleotide sequence from the analyte/probe complex,
(c) contacting the probe assembly to the analyte under conditions effective to promote binding of the capture oligonucleotide sequence to the target portion of the analyte, so as to form an analyte/probe complex; and
(d) contacting the analyte/probe complex; to the analyte to the nuclease under conditions effective to promote nuclease activity, so as to degrade the double stranded portion of the probe assembly so as to release at least the reporter oligonucleotide sequence from the analyte/probe complex
(e) detecting the presence of at least the released reporter oligonucleotide sequence so as to determine the presence of the analyte in a sample.

37. The method of claim 36, wherein the analyte includes a polynucleotide and the capture sequence includes a sequence complementary to a corresponding target sequence of the analyte.

38. The method of claim 36, wherein the analyte includes a non-nucleotide biopolymer, and the capture sequence includes an aptamer having a specific binding capability for the target portion of the analyte, and wherein the binding of the aptamer to the biomolecule induces a change to the aptamer subjecting the double stranded portion of the probe assembly to degradation by the nuclease so as to release at least the reporter oligonucleotide sequence from the analyte/probe complex.
